(19) United States
(12) Patent Application Publication
(10) Pub. No.: US 2026/0263470 A1
(43) Pub. Date: Sep. 10, 2026

(54) A METHOD FOR THE TREATMENT OF CHRONIC HAND ECZEMA IN PATIENTS WITH MODERATE TO SEVERE CHRONIC HAND ECZEMA

(71) Applicant: LEO Pharma A/S, Ballerup (DK)

(72) Inventors: Klaus NIELSEN, Ballerup (DK); Lucine LEHMANN, Ballerup (DK)

(21) Appl. No.: 19/165,568

(22) PCT Filed: Mar. 13, 2024
(86) PCT No.: PCT/EP2024/056617

§ 371 Date: Sep. 15, 2025

(30) Foreign Application Priority Data
Mar. 17, 2023 (EP) 23162528.6
May 8, 2023 (EP) 23172110.1
Sep. 5, 2023 (EP) 23195419.9
Oct. 26, 2023 (EP) 23206222.4
Dec. 7, 2023 (EP) 23215005.2

(51) Int. Cl. A61K 31/519; A61P 17/00; A61P 17/04

(52) U.S. Cl. CPC A61K 31/519; A61P 17/00; A61P 17/04

(57) ABSTRACT

A method of treating moderate to severe chronic hand eczema in a human patient comprising twice daily administration to the skin of said human patient in need thereof of an acidified aqueous topical composition comprising comprises 20 mg/g delgocitinib on a free base basis or a pharmaceutically acceptable salt.

The present invention relates to method of treating chronic hand eczema in a human patient comprising administering to the skin of said human patient an acidified aqueous topical formulation comprising delgocitinib.

### BACKGROUND

Chronic Hand Eczema (CHE) is a serious inflammatory skin disorder located anywhere on the hands or wrists. It is clinically characterised by erythema, infiltration, hyperkeratosis, oedema, and vesicles. Secondary signs include scaling, fissures, and erosions, and the condition may be exacerbated by bacterial infections. Important symptoms include itch and pain, and the disease is often characterised by chronic relapses and a poor prognosis.

CHE refers to hand eczema which persists for more than 3 months or returns twice or more often within 12 months.

Hand eczema is usually multifactorial, and it is generally agreed that no simple relationships exist between clinical patterns and aetiological diagnoses (J Eur Acad Dermatol Venereol. 2015; 29(12):2417-2422).

Several different classifications have been proposed (Diepgen et al., J Dtsch Dermatol Ges. 2015; 13(1):e1-22, Cronin E et al., Contact Dermatitis. 1985; 13(3):153-161, Johansen et al. Contact Dermatitis. 2011; 65(1):13-21) and the most common subtypes of CHE are found to be irritant contact dermatitis, atopic hand eczema, and hyperkeratotic eczema (Apfelbacher et al. Acta Derm Venereol. 2014; 94(2):163-167).

Other subtypes include allergic contact dermatitis, contact urticaria/protein contact dermatitis, and recurrent vesicular hand eczema (pompholyx).

The reported prevalence and incidence rates of hand eczema vary considerably, depending on the methodology in the collection of data. In a review of data available from 1964 to 2007, the point prevalence of hand eczema in the general population was approximately 4%, 1-year prevalence about 10%, and life-time prevalence approached 15%, approximately 7-10% of patients with hand eczema reported symptoms ‘nearly all the time’, implying a chronic state of the disease. Based on data from 7 studies, the incidence rate of hand eczema was 5.5 cases/1000 person-years with a higher median incidence rate among women (Diepgen et al., J Dtsch Dermatol Ges. 2015; 13(1):e1-22). Several risk factors, such as pre-existing AD, female sex, wet work, and contact allergy have been identified (Thyssen et al., Contact Dermatitis. 2010; 62(2):75-87, Mortz el al., Br J Dermatol. 2014; 171(2):313-323).

The prevalence of hand eczema is different across age groups (6) with a mean/median first onset in the early or mid-20'ies (Anveden et al., Contact Dermatitis. 2006; 54(5):272-277, Hald et al., Br J Dermatol. 2008; 158(4):773-777, Lind et al., Occup Environ Med. 2007; 64(3):191-195). However, approximately one-third of men and women report their first hand eczema before the age of 20 (Meding B et al., J Invest Dermatol. 2004; 122(4):873-877).

Although the molecular mechanisms underlying CHE are not fully understood, a large panel of cytokine-mediated signalling cascades have been identified as part of the pathophysiology, including cytokine responses representing Th2 pathway (IL-4, IL-13), Th22 pathway (IL-22), Th17 pathway (IL-17), Th1 pathway (interferon-γ), and the JAK/STAT pathway. As the JAK proteins are required for signalling of most cytokines, blocking of JAKs reduces cytokine signalling and thereby abrogates the vicious cycle that leads to the development of CHE (Pedersen et al., J Invest Dermatol. 2007; 127(11):2585-2595, Brunner et al., J Allergy Clin Immunol. 2017; 139(4S):S65-S76, Gittler et al., Journal of Allergy and Clinical Immunology. 2013; 131(2):300-313).

CHE is generally difficult to treat and presents with periods of flares and periods of remissions.

Treatment of CHE involves different disease management strategies such as elimination of triggers, general skin care, and anti-inflammatory therapy in a step-wise approach. General skin care in terms of emollients is widely used and recommended by physicians, but evidence of efficacy is sparse. Elimination of triggers such as allergens and irritants can be effective and a necessary prerequisite for successful therapy on a longer term, but elimination may in many circumstances be difficult to achieve. Although lacking documented treatment effect, TCS remain the mainstay of topical anti-inflammatory therapy for hand eczema. However, long-term use of TCS is restricted due to side effects such as skin atrophy and potential inhibition of skin barrier repair.

Whereas mild CHE to some extent may be managed by elimination of triggers and general skin care, management of moderate to severe CHE is more cumbersome. Alitretinoin is the only approved product specifically indicated for treatment of CHE but is only indicated for severe CHE and only approved in few countries worldwide. Alitretinoin treatment has various safety limitations and is therefore only indicated for use in adults who have severe CHE that is unresponsive to treatment with potent TCS.

Considering the paucity of approved therapies for the treatment of CHE, other therapeutic options are limited to those approved for other skin diseases with an inflammatory pathophysiology. These applied treatments lack the clinical documentation for use in CHE and are restricted to short-term use which is not suitable in a chronic disorder characterised by relapsing features often resulting in long-term treatment exposure.

As the currently available treatment options either lack documented treatment effect or are limited by restrictions of long-term use due to safety concerns, there is a high unmet medical need for new topical treatment of moderate to severe CHE with high efficacy in combination with a good safety profile especially for long-term use. New and better treatments would potentially improve the everyday lives of patients with moderate to severe CHE. Delgocitinib has the potential to address the unmet medical need associated with this serious disease.

Delgocitinib is disclosed in WO2011013785 and has the general formula

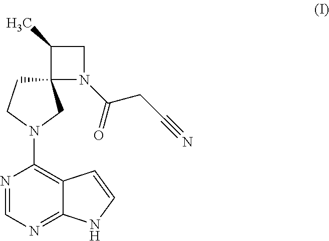

and is suitably prepared by one of the methods disclosed in WO2018117152. Delgocitinib is a pan-JAK inhibitor, which blocks various cytokine-mediated signalling pathways and widely suppresses the activation of immune and inflammatory cells such as T-cells, B-cells, mast cells, and monocytes activated by these cytokines

The efficacy and safety of delgocitinib in mild to severe CHE has been demonstrated in a phase 2a trial with delgocitinib 30 mg/g and in a phase 2b dose-ranging trial with delgocitinib (1, 3, 8, and 20 mg/g) (Worm et al., British Journal of Dermatology (2022), 187, p-42-51 and British Journal of Dermatology (May 2020, 185, (5), 1103-1110).

There is a clear unmet need for new long-term treatment options in the treatment of patients suffering from moderate to severe CHE.

It has now been found that and acidified aqueous composition comprising delgocitinib has significant effect in patients suffering from moderate to severe chronic hand eczema (CHE).

### SUMMARY OF THE INVENTION

The present invention relates to a method of treating moderate to severe chronic hand eczema in a human patient in need thereof comprising twice daily administration to the skin of said human patient, of an acidified aqueous topical composition comprising comprises 20 mg/g delgocitinib on a free base basis or a pharmaceutically acceptable salt thereof, wherein the patient has disease severity graded as moderate to severe at screening and baseline according to IGA-CHE (i.e. an IGA-CHE score of 3 or 4).

In another aspect the invention relates to said acidified aqueous topical composition for use in a method of treating moderate to severe chronic hand eczema.

### DETAILED DESCRIPTION OF THE INVENTION

### Figures

FIG. 1: IGA-CHE TS responders from week 0 to 16. a) IGA-CHE TS by visit, b) TS defined as an IGACHE score of 0 (clear) or 1 (almost clear) with a ≥2step improvement from baseline

FIGS. 2A, 2B and 2C: HECSI75 as percentage responders from week 0 to week 16 (2A), HECSI90 and DLQI≥4 as percentage responders from week 0 to week 16 (2B) and HECSI75, HECSI90 and DLQI 4 as percentage of responders at week 16 (2C).

FIG. 3. Change in HESD pain score (weekly average) from baseline to Week 16 in DELTA 1 and DELTA 2 (full analysis set, WOCF). HESD pain score was reported daily by study participants and ranges from 0 (‘no symptom’) to 10 (‘severe symptom’).

FIGS. 4A and B. Proportion of patients achieving (A) HESD pain ≥4-point reduction and (B) HESD itch ≥4-point reduction from baseline to Week 16 among study participants with a corresponding baseline HESD pain/itch score (weekly average) of ≥4 points in DELTA 1 and DELTA 2 (full analysis set, NRI) (A) HESD pain ≥4-point reduction. FIG. 4(B) HESD itch ≥4-point reduction. HESD itch and pain scores were reported daily by study participants and range from 0 (‘no symptom’) to 10 (‘severe symptom’).

FIG. 5. Change in HESD itch score (weekly average) from baseline to Week 16 in DELTA 1 and DELTA 2 (full analysis set, WOCF). HESD itch score was reported daily by study participants and ranges from 0 (‘no symptom’) to 10 (‘severe symptom’).

FIG. 6. Summary of adverse events in DELTA 1 and DELTA 2 (safety analysis set). AEs with start date on or after the date of first study treatment application or with a worsening in severity after first study treatment application are included. Classification is according to MedDRA version 24.0. ^{a}Reporting exacerbation or worsening of CHE, which exceeded normal fluctuation or appeared in areas not normally affected by CHE.

FIG. 7. Outcomes and action taken with study drug following occurrence of adverse events in DELTA 1 and DELTA 2 (safety analysis set).

FIG. 8. Relative (% of positive control) individual and mean effect of ointment and cream application to human skin explants (NativeSkin) at 24 hours after treatment. The different colors represent the different donors, for each treatment.

FIG. 9. Open flow micro-perfusion probe inserted in dermis.

FIG. 10. Changes from baseline in EQ-5D at week 16 in all patients and by treatment response as assessed by HECSI, IGA-CHE and HEDS itch and pain scores.

FIG. 11. Changes from baseline in DLGQI at week 16 in all patients and by treatment response as assessed by HECSI, IGE-CHE and HEDS itch and pain scores.

FIG. 12. Proportion of patients who achieved HESD itch ≥4-point improvement, and HESD pain ≥4-point improvement over time—pooled data from DELTA 1 and DELTA 2. *Statistically significant versus vehicle with adjustment for multiplicity ** Statistically significant versus vehicle without adjustment for multiplicity Based on the number of patients whose baseline value was ≥4 (scale from 0-10).

### ABBREVIATIONS

AE=adverse event;
CHE=chronic hand eczema;
cDLQI=Children's Dermatology Life Quality Index;
HECSI=Hand Eczema Severity Index;
HECSI-75=at least 75% improvement in HECSI score from baseline
HECSI-90=at least 90% improvement in HECSI score from baseline;
HESD=Hand Eczema Symptom Diary©.
IE=intercurrent event;
IGA-CHE=Investigator's Global Assessment for chronic hand eczema©;
IGA-CHE TS=IGA-CHE treatment success, i.e. an IGA-CHE score of 0 (clear) or 1 (almost clear) with a ≥2-step improvement from baseline;
TS=treatment success.
DLQI=Dermatology Life Quality Index;
EQ-5D-5L=EuroQol 5-Dimension Health Questionnaire 5 Level;
HEIS=Hand Eczema Impact Scale;
PDAL=Proximal Daily Activity Limitations
WPAI:CHE=Work Productivity and Activity Impairment: Chronic Hand Eczema.
cDLQI=Children's Dermatology Life Quality Index;
AS=area score; SS=severity score;
CI, confidence interval;
LS, least squares;
SE, standard error;
WOCF, worst observation carried forward;
NRI, non-responder imputation;
E, number of events;
N, number of patients in analysis set;
n, number of patients with observation;
PYO, patient years of observation; R, rate calculated as (E/PYO)*100.

As used herein in connection with a value or amount about means+/−10% of the value or amount.

### Measurements of Efficacy

### IGA-CHE

The IGA-CHE rates the severity of the subject's global disease and is based on a 5-point scale ranging from 0 (clear) to 4 (severe) (Table 1). The assessment will be based on the condition of the disease at the time of evaluation and not in relation to the condition at a previous visit. New lesions that occur on previously untreated areas will be included in the assessment.

TABLE 1

| IGA-CHE | IGA-CHE | |
|---|---|---|
| severity | score | Sign and intensity |
| Clear | 0 | No signs of erythema, scaling, hyperkeratosis/lichenification, |
| | | vesiculation, oedema or fissures |
| Almost | 1 | Barely perceptible erythema |
| clear | | No signs of scaling, hyperkeratosis/ lichenification, |
| | | vesiculation, oedema or fissures |
| Mild | 2 | At least one: |
| | | Slight but definite erythema (pink) |
| | | Slight but definite scaling (mostly fine scales) |
| | | Slight but definite hyperkeratosis/lichenification |
| | | and at least one: |
| | | Scattered vesicles, without erosion |
| | | Barely palpable oedema |
| | | Superficial fissures |
| Moderate | 3 | At least one: |
| | | Clearly perceptible erythema (dull red) |
| | | Clearly perceptible scaling (coarse scales) |
| | | Clearly perceptible hyperkeratosis/lichenification |
| | | and at least one: |
| | | Clustered vesicles, without visible erosion |
| | | Definite oedema |
| | | Definite fissures |
| Severe | 4 | At least one: |
| | | Marked erythema (deep or bright red) |
| | | Marked and thick scaling |
| | | Marked hyperkeratosis/lichenification |
| | | and at least one: |
| | | High density of vesicles with erosions |
| | | Marked oedema |
| | | One or more deep fissures |

### Hand Eczema Severity Index (HECSI)

The HECSI is an instrument used in clinical trials to rate the severity of 6 clinical signs (erythema, infiltration/papulation, vesicles, fissures, scaling, and oedema) and the extent of the lesions on each of the 5 hand regions (fingertips, fingers [except fingertips], palm of hands, back of hands, and wrists) by use of standard scales (Held et al., Br J Dermatol. 2005; 152(2):302-307).

For each hand region (total of both hands, e.g. 10 fingers), the investigator rates the average severity of each of the 6 clinical signs of hand eczema using a 4-point severity scale ranging from 0 (none/absent) to 3 (severe) (Table 2). The investigator also rates the extent of the lesions by assessing the percentage of the hand regions these lesions occupy and converting it to a score based on a 5-point scale (the area score) (Table 2). For each of the hand regions, the region score will be calculated by adding up the severity scores for the 6 clinical signs of hand eczema and multiplying with the area score (Table 3). The HECSI score equals the sum of the region scores and will range from 0 (lowest possible score) to 360 (highest possible score).

The assessment will be based on the condition of the disease at the time of evaluation and not in relation to the condition at a previous visit. New CHE lesions that occur on previously untreated areas will be included in the assessment.

TABLE 2
HECSI severity score scale and area score scale

| Severity score (SS) scale | Area score (AS) scale (based on |
|---|---|
| (based on both hands) | the affected area of both hands) |

| | | | |
|---|---|---|---|
| 0 | None/absent | 0 | 0% affected area |
| 1 | Mild | 1 | 1% to 25% affected area |
| 2 | Moderate | 2 | 26% to 50% affected area |
| 3 | Severe | 3 | 51% to 75% affected area |
| | | 4 | 76% to 100% affected area |

TABLE 3
Calculation of the total HECSI score

| | | Infiltration/ | | | | | Area | |
|---|---|---|---|---|---|---|---|---|
| Hand region | Erythema | papulation | Vesicles | Fissures | Scaling | Oedema | score | Score |
| Fingertips | (SS+ | SS+ | SS+ | SS+ | SS+ | SS) | ×AS | |
| Fingers (except | (SS+ | SS+ | SS+ | SS+ | SS+ | SS) | ×AS | |
| fingertips) | | | | | | | | |
| Palm of hands | (SS+ | SS+ | SS+ | SS+ | SS+ | SS) | ×AS | |
| Back of hands | (SS+ | SS+ | SS+ | SS+ | SS+ | SS) | ×AS | |
| Wrists | (SS+ | SS+ | SS+ | SS+ | SS+ | SS) | ×AS | |

| The total HECSI score equals the sum of the 5 above region scores: | (̅r̅a̅n̅g̅e̅ 0̅-̅ |
|---|---|
| | 360) |

### Patient-Reported Outcomes (Efficacy)

The PRO HESD is considered an efficacy assessment

### Hand Eczema Symptom Diary (HESD)

The HESD is a 6-item PRO instrument designed to assess severity of CHE signs and symptoms. Subjects will assess the worst severity of 6 individual signs and symptoms of CHE (itch, pain, cracking, redness, dryness, and flaking) over the past 24 hours using an 11-point numeric rating scale with anchors of 0=‘no (symptom)’ and 10=‘severe (symptom)’. The HESD score is derived as an average of the 6 items.

### Safety Assessments

### Patient-Reported Outcomes (Health-Related Quality of Life and Work Productivity)

### Patient Global Impression of Severity (PGI-S) Questionnaires

The PGI-S is a 1-item questionnaire designed to assess the subject's global perception of their itch, pain, or chronic hand eczema signs and symptoms over the past week on a 4-point categorical response scale (‘none’, ‘mild’, ‘moderate’, or ‘severe’), Table 4.

TABLE 4
Patient Global Impression of Severity (PGI-S) questionnaires

| Itch PGI-S | Pain PGI-S | HESD PGI-S |
|---|---|---|
| Please choose the | Please choose the | Please choose the |
| response below that best | response below that best | response below that best |
| describes the severity of | describes the severity of | describes the severity of |
| any itch on your hands | any pain on your hands | your hand eczema signs |
| over the past week | over the past week | and symptoms over the |
| | | past week |
| Response scale: | Response scale: | Response scale: |
| None | None | None |
| Mild | Mild | Mild |
| Moderate | Moderate | Moderate |
| Severe | Severe | Severe |

### Patient Global Impression of Change (PGI-C) Questionnaires

The PGI-C is a 1-item questionnaire designed to assess the subject's impression of changes (38). From 5 response options (‘much better’, ‘a little better’, ‘no change’, ‘a little worse’, or ‘much worse’), the subjects have to select the one response that best describes the overall change in their itch, pain, or chronic hand eczema signs and symptoms since they started IMP treatment, Table 5.

TABLE 5
Patient Global Impression of Change (PGI-C) questionnaires

| Itch PGI-C | Pain PGI-C | HESD PGI-C |
|---|---|---|
| Please choose the | Please choose the | Please choose the |
| response below that best | response below that best | response below that best |
| describes the overall | describes the overall | describes the overall |
| change in any itch on | change in any pain on | change in your hand |
| your hands since you | your hands since you | eczema signs and |
| started taking the study | started taking the study | symptoms since you |
| medication | medication | started taking the study |
| | | medication |
| Response scale: | Response scale: | Response scale: |
| Much better | Much better | Much better |
| A little better | A little better | A little better |
| No change | No change | No change |
| A little worse | A little worse | A little worse |
| Much worse | Much worse | Much worse |

### Hand Eczema Impact Scale (HEIS)

HEIS includes 9 items addressing the subject's perception of the impact of hand eczema on their daily activities, embarrassment, frustration, sleep, work, and physical functioning over the past 7 days. Each item is scored on a 5-point scale (0=‘not at all’, 1=‘a little’, 2=‘moderately’, 3=‘a lot’, 4=‘extremely’). The HEIS score is the average of the 9 items. The highest possible score is 4, and a high score is indicative of a high impact. 6 domain scores can be calculated for HEIS: PDAL (average of 3 items), embarrassment with the appearance of the hands (average of 2 items), frustration with CHE (1 item), sleep (1 item), work (1 item), and physical functioning (1 item).

### Patient's Global Assessment of Disease Severity (PaGA)

Subjects will make a global assessment of the severity of their hand eczema. The assessment will be made using a 5-point scale (0=‘clear’, 1=‘almost clear’, 2=‘mild’, 3=‘moderate’, 4=‘severe’) and will be based on the severity of their hand eczema at the time of the assessment, Table 6.

TABLE 6
Patient's Global Assessment of disease severity (PaGA)

Rate the severity of your hand eczema right now. In making your rating you
should think about the severity of any hand eczema symptoms you have right
now, including scaling, blistering, cracking, swelling, redness, or thickening of
the skin on your hands.
Clear (No hand eczema symptoms)
Almost clear (Only slight redness, no other hand eczema symptoms)
Mild
Moderate
Severe

### Dermatology Life Quality Index (DLQI)

The DLQI is a validated questionnaire with content specific to those with dermatologic conditions. It consists of 10 items addressing the subject's perception of the impact of their skin disease on different aspects of their quality of life over the last week, such as dermatology-related symptoms and feelings, daily activities, leisure, work or school, personal relationships, and the treatment (39). Each item is scored on a 4-point Likert scale (0=‘not at all/not relevant’; 1=‘a little’; 2=‘a lot’; 3=‘very much’). The DLQI score is the sum of the 10 items (score ranging from 0 to 30); a high score is indicative of a poor quality of life.

The cDLQI is a validated questionnaire with content specific to those with dermatologic conditions. It consists of 10 items addressing the subject's perception of the impact of their skin disease on different aspects of their quality of life over the last week such as dermatology-related symptoms and feelings, friendships, leisure, school or holiday, adverse comments, sleep, and the treatment (35). Each item is scored on a 4-point Likert scale (0=‘not at all/not relevant’; 1=‘a little’; 2=‘a lot’; 3=‘very much’). The total score is the sum of the 10 items (0 to 30); a high score is indicative of a poor quality of life.

### EuroQol 5-Dimension Health Questionnaire 5 Level (EQ-5D-5L)

The EQ-5D-5L is a standardised measure of health status developed by the EuroQol group to provide a simple, generic measure of health for clinical and economic appraisal (EuroQol—a new facility for the measurement of health-related quality of life. Health Policy. 1990; 16(3):199-208).

The EQ-5D-5L is a self-administered questionnaire used to assess health status ‘today’ and is divided into 2 sections.

The first section includes 5 dimensions (mobility, self-care, usual activity, pain/discomfort, and anxiety/depression). Each dimension will be assessed by the subject using a 5-point scale (‘no problems’, ‘slight problems’, ‘moderate problems’, ‘severe problems’, and ‘unable to/extreme problems’). The EQ-5D-5L index score is derived from the 5 dimensions and has been converted from the 5L system to the 3L system using the EQ-5D-5L crosswalk value set. The index score ranges from −0.594 to 1.0 (based on the UK country-specific value set), with a higher score indicating a better health status.

The second section consists of a vertical visual analogue scale anchored at 0 (‘the worst health you can imagine’) and 100 (‘the best health you can imagine’). Work Productivity and Activity Impairment: Chronic Hand Eczema (WPAI:CHE)

The impact of CHE on the subject's ability to work and perform regular activities will be assessed by WPAI:CHE, which is an instrument to measure impairments in both paid work and unpaid work (Reilly et al., Pharmacoeconomics. 1993; 4(5):353-365). The WPAI:CHE consists of 6 items, and scores can be calculated for 4 domains, each reflecting the percentage impairment due to CHE during the past 7 days, with higher numbers indicating greater impairment and less productivity:
Absenteeism: percentage work time missed due to CHE for those who were currently employed.
Presenteeism: percentage impairment while working due to CHE for those who were currently employed and actually worked in the past 7 days.
Work productivity loss: percentage overall work impairment due to CHE for those who were currently employed.
Activity impairment: percentage activity impairment due to CHE for all respondents.

Endpoints to be investigated are shown in table 7 and 8 below.

TABLE 7
Endpoints

Primary endpoint:
IGA-CHE TS at Week 16.
Key secondary endpoints:
HECSI-75 at Week 16.
HECSI-75 at Week 8.
HECSI-90 at Week 16.
IGA-CHE TS at Week 8.
IGA-CHE TS at Week 4.
Key secondary endpoint:
Percentage change in HECSI score from baseline to Week 16.
Key secondary endpoints:
Reduction of HESD itch score (weekly average) of ≥4 points from baseline at
Week 16. Among subjects with a baseline HESD itch score (weekly average) ≥4
points.
Reduction of HESD itch score (weekly average) of ≥4 points from baseline at
Week 8. Among subjects with a baseline HESD itch score (weekly average) ≥4
points.
Reduction of HESD itch score (weekly average) of ≥4 points from baseline at
Week 4. Among subjects with a baseline HESD itch score (weekly average) ≥4
points.
Reduction of HESD itch score (weekly average) of ≥4 points from baseline at
Week 2. Among subjects with a baseline HESD itch score (weekly average) ≥4
points.
Reduction of HESD score (weekly average) of ≥4 points from baseline at
Week 16. Among subjects with a baseline HESD score (weekly average) ≥4
points.
Reduction of HESD score (weekly average) of ≥4 points from baseline at
Week 8. Among subjects with a baseline HESD score (weekly average) ≥4
points.
Reduction of HESD score (weekly average) of ≥4 points from baseline at
Week 4. Among subjects with a baseline HESD score (weekly average) ≥4
points.
Reduction of HESD pain score (weekly average) of ≥4 points from baseline at
Week 16. Among subjects with a baseline HESD pain score (weekly average) ≥4
points.
Reduction of HESD pain score (weekly average) of ≥4 points from baseline at
Week 8. Among subjects with a baseline HESD pain score (weekly average) ≥4
points.
Reduction of HESD pain score (weekly average) of ≥4 points from baseline at
Week 4. Among subjects with a baseline HESD pain score (weekly average) ≥4
points.
Reduction of DLQI score of ≥4 points from baseline at Week 16. Among
subjects with a baseline DLQI score ≥4 points
Key secondary endpoints:
Change in HESD itch score (weekly average) from baseline to Week 16.
Change in HESD score (weekly average) from baseline to Week 16.
Change in HESD pain score (weekly average) from baseline to Week 16.
Change in HEIS score from baseline to Week 16.
Change in HEIS PDAL score from baseline to Week 16.
Change in DLQI score from baseline to Week 16.

TABLE 8
Secondary and exploratory endpoints

Secondary endpoint
Number of treatment emergent AEs from baseline up to Week 16
(Week 18 for subjects not participating in the LTE trial) per subject. An
event will be considered treatment emergent if started after the first
application of IMP or if started before the first application of IMP and
worsened in severity after first dose of IMP.
Exploratory endpoints
Efficacy
IGA-CHE TS at Weeks 1, 2, and 12.
HECSI-75 at Weeks 1, 2, 4, and 12.
HECSI-90 at Weeks 1, 2, 4, 8, and 12.
Percentage change in HECSI score from baseline to Weeks 1, 2, 4, 8,
and 12.
Reduction of HESD itch score (weekly average) of ≥4 points from
baseline at Weeks 1 and 12. Among subjects with a baseline HESD itch
score (weekly average) ≥4 points.
Reduction of HESD itch score (weekly average) of ≥3 points from
baseline at Weeks 1, 2, 4, 8, 12, and 16. Among subjects with a
baseline HESD itch score (weekly average) ≥3 points.
Time to reduction of HESD itch score (weekly average) of ≥4 points.
Among subjects with a baseline HESD itch score (weekly average) ≥4
points.
Change in HESD itch score (weekly average) from baseline to Weeks 1,
2, 4, 8, and 12.
Reduction of HESD score (weekly average) of ≥4 points from baseline
at Weeks 1, 2, and 12. Among subjects with a baseline HESD score
(weekly average) ≥4 points.
Reduction of HESD score (weekly average) of ≥3 points from baseline
at Weeks 1, 2, 4, 8, 12, and 16. Among subjects with a baseline HESD
score (weekly average) ≥3 points.
Time to reduction of HESD score (weekly average) of ≥4 points. Among
subjects with a baseline HESD score (weekly average) ≥4 points.
Change in HESD score (weekly average) from baseline to Weeks 1, 2,
4, 8, and 12.
Reduction of HESD pain score (weekly average) of ≥4 points from
baseline at Weeks 1, 2, and 12. Among subjects with a baseline HESD
pain score (weekly average) ≥4 points.
Reduction of HESD pain score (weekly average) of ≥3 points from
baseline at Weeks 1, 2, 4, 8, 12, and 16. Among subjects with a
baseline HESD pain score (weekly average) ≥3 points.
Time to reduction of HESD pain score (weekly average) of ≥4 points.
Among subjects with a baseline HESD pain score (weekly average) ≥4
points.
Change in HESD pain score (weekly average) from baseline to Weeks
1, 2, 4, 8, and 12.
Change in HESD (weekly average for each individual symptom
[excluding itch and pain]) score from baseline to Week 16.
Health-related quality of life and efficacy
Change in HEIS score from baseline to Weeks 1, 2, 4, 8, and 12.
Reduction of HEIS score of ≥1.5 points at Weeks 1, 2, 4, 8, 12, and 16.
Among subjects with a baseline HEIS score ≥1.5 points.
Change in HEIS PDAL score from baseline to Weeks 1, 2, 4, 8, and 12.
Reduction of HEIS PDAL score of ≥1.5 points at Weeks 1, 2, 4, 8, 12,
and 16. Among subjects with a baseline HEIS PDAL score ≥1.5 points.
Change in HEIS (each individual domain [excluding PDAL]) score from
baseline to Weeks 1, 2, 4, 8, 12, and 16.
Reduction in DLQI score of ≥4 points at Weeks 1, 4, 8, and 12. Among
subjects with a baseline DLQI score ≥4 points.
Change in DLQI score from baseline to Weeks 1, 4, 8, and 12.
Change in EQ-5D-5L index score from baseline to Weeks 1, 4, 8, 12,
and 16.
Change in EQ-5D-5L visual analogue scale score from baseline to
Weeks 1, 4, 8, 12, and 16.
Change in WPAI:CHE absenteeism score from baseline to Week 4, 8,
and 16. Among subjects with paid work at baseline.
Change in WPAI:CHE presenteeism score from baseline to Week 4, 8,
and 16. Among subjects with paid work at baseline.
Change in WPAI:CHE work productivity loss score from baseline to
Week 4, 8, and 16. Among subjects with paid work at baseline.
Change in WPAI:CHE activity impairment score from baseline to Week
4, 8, and 16.
Change in gene expression measured in skin tape strips from baseline
to Week 16.

TABLE 9
Primary, key secondary, and secondary endpoints

Endpoints
Primary endpoint:
IGA-CHE TS at Week 16.
(IGA-CHE TS refers to a score of 0 [clear] or 1 [almost clear] with a ≥2-step
improvement from baseline).
Key secondary endpoint:
HECSI-90 at Week 16.
Secondary endpoint:
IGA-CHE TS at Weeks 2, 4, 8, and 12.
Key secondary endpoints:
Reduction of HESD itch score (weekly average) of ≥4 points from baseline at
Week 16. Among subjects with a baseline HESD itch score (weekly average) ≥4
points
1) Reduction of HESD pain score (weekly average) of ≥4 points from baseline at
Week 16. Among subjects with a baseline HESD pain score (weekly average) ≥4
points.
2) Reduction of HESD score (weekly average) of ≥4 points from baseline at
Week 16. Among subjects with a baseline HESD score (weekly average) ≥4
points.
Secondary endpoint:
Change in cDLQI score from baseline to Week 16.

TABLE 10
Secondary and exploratory endpoints

Secondary endpoint
1) Number of treatment-emergent AEs from baseline up to Week 18. An event will
be considered treatment emergent if started after the first application of IMP or if
started before the first application of IMP and worsened in severity after first dose
of IMP.
exploratory endpoints
Efficacy
HECSI-90 at Weeks 4 and 8.
HECSI-75 at Weeks 4, 8, and 16.
Percentage change in HECSI score from baseline to Weeks 4, 8, and 16.
Health-related quality of life and efficacy
Reduction of HESD itch score (weekly average) of ≥4 points from baseline at
Weeks 2, 4, and 8. Among subjects with a baseline HESD itch score (weekly
average) ≥4 points.
Reduction of HESD itch score (weekly average) of ≥3 points from baseline at
Weeks 2, 4, 8, and 16.4. Among subjects with a baseline HESD itch score
(weekly average) ≥3 points.
Change in HESD itch score (weekly average) from baseline to Weeks 2, 4, 8,
and 16.
Reduction of HESD pain score (weekly average) of ≥4 points from baseline at
Weeks 2, 4, and 8. Among subjects with a baseline HESD pain score (weekly
average) ≥4 points.
Reduction of HESD pain score (weekly average) of ≥3 points from baseline at
Weeks 2, 4, 8, and 16. Among subjects with a baseline HESD pain score
(weekly average) ≥3 points
Change in HESD pain score (weekly average) from baseline to Weeks 2, 4, 8,
and 16.
Reduction of HESD score (weekly average) of ≥4 points from baseline at
Weeks 2, 4, and 8. Among subjects with a baseline HESD score (weekly
average) ≥4 points
Reduction of HESD score (weekly average) of ≥3 points from baseline at
Weeks 2, 4, 8, and 16. Among subjects with a baseline HESD score (weekly
average) ≥3 points
Change in HESD score (weekly average) from baseline to Weeks 2, 4, 8,
and 16.
Change in cDLQI score from baseline to Week 4.

As described above, the invention relates to: A method of treating moderate to severe chronic hand eczema in a human patient comprising twice daily administration to the skin of said human patient in need thereof of an acidified aqueous topical composition comprising comprises 20 mg/g delgocitinib on a free base basis or a pharmaceutically acceptable salt thereof, wherein the patient has disease severity graded as moderate to severe at screening and baseline according to IGA-CHE (i.e. an IGA-CHE score of 3 or 4).

In a further embodiment the invention relates to: Use of an acidified aqueous topical composition comprising comprises 20 mg/g delgocitinib on a free base basis or a pharmaceutically acceptable salt thereof for treating moderate to severe chronic hand eczema in a human patient comprising twice daily administration to the skin of said human patient in need thereof, wherein the patient has disease severity graded as moderate to severe at screening and baseline according to IGA-CHE (i.e. an IGA-CHE score of 3 or 4).

In a further embodiment the invention relates to: The use of delgocitinib for the manufacture of an acidified aqueous topical composition comprising comprises 20 mg/g delgocitinib on a free base basis or a pharmaceutically acceptable salt thereof for treating moderate to severe chronic hand eczema in a human patient comprising twice daily administration to the skin of said human patient in need thereof, wherein the patient has disease severity graded as moderate to severe at screening and baseline according to IGA-CHE (i.e. an IGA-CHE score of 3 or 4).

In a further embodiment, the invention relates to the method, use, or the use above, wherein the patient has a HESD itch score (weekly average) of ≥4 points at baseline.

In a further embodiment, the invention relates to the method, use, or the use above, wherein the patient: is an adolescent aged 12 to 17 or an adult aged 18 years or above; wherein the patient have hand eczema that has persisted for more than 3 months or returned twice or more within the last 12 months.

In a further embodiment, the invention relates to the method, use, or the use above, wherein the patient has a documented recent history of inadequate response to treatment with topical corticosteroids (TCS) or for whom TCS are documented to be otherwise medically inadvisable (e.g. due to important side effects or safety risks).

In a further embodiment, the invention relates to the method, use, or the use above, wherein the patient achieves an IGA-CHE score of 0 (clear) or 1 (almost clear) with a ≥2-step improvement from baseline at week 16.

In a further embodiment, the invention relates to the method, use, or the use above, wherein the patient achieves an IGA-CHE score of 0 (clear) or 1 (almost clear) with a ≥2-step improvement from baseline at week 12.

In a further embodiment, the invention relates to the method, use, or the use above, wherein the patient achieves an IGA-CHE score of 0 (clear) or 1 (almost clear) with a ≥2-step improvement from baseline at week 8.

In a further embodiment, the invention relates to the method, use, or the use above, wherein the patient achieves an IGA-CHE score of 0 (clear) or 1 (almost clear) with a ≥2-step improvement from baseline at week 4.

In a further embodiment, the invention relates to the method, use, or the use above, wherein the patient achieves an IGA-CHE score of 0 (clear) or 1 (almost clear) with a ≥2-step improvement from baseline at week 2.

In a further embodiment, the invention relates to the method, use, or the use above, wherein the patient achieves an IGA-CHE score of 0 (clear) or 1 (almost clear) with a ≥2-step improvement from baseline at week 1.

In a further embodiment, the invention relates to the method, use, or the use above, wherein the patient achieves at least 90% improvement in HECSI score from baseline at week 16, or week, 8 or week 4, week 2 or week 1.

In a further embodiment, the invention relates to the method, use, or the use above, wherein the patient achieves at least 75% improvement in HECSI score from baseline at week 16, or week 8, or week 4, or week 2 or week 1.

In a further embodiment, the invention relates to the method, use, or the use above, wherein the patient has a baseline HESD score (weekly average) ≥4 points and achieves a reduction of HESD score (weekly average) of ≥4 points from baseline at week 16.

In a further embodiment, the invention relates to the method, use, or the use above, wherein the patient has a baseline HESD score (weekly average) ≥4 points and achieves a reduction of HESD score (weekly average) of ≥4 points from baseline at week 12.

In a further embodiment, the invention relates to the method, use, or the use above, wherein the patient has a baselined HESD score (weekly average ≥4 points at baseline and achieves a reduction of HESD score (weekly average) of ≥4 points from baseline at Week 8.

In a further embodiment, the invention relates to the method, use, or the use above, wherein the patient has a baselined HESD score (weekly average ≥4 points at baseline and achieves a reduction of HESD score (weekly average) of ≥4 points from baseline at Week 4.

In a further embodiment, the invention relates to the method, use, or the use above, wherein the patient has a baselined HESD score (weekly average ≥4 points at baseline and achieves a reduction of HESD score (weekly average) of ≥4 points from baseline at Week 2.

In a further embodiment, the invention relates to the method, use, or the use above, wherein the patient has a baselined HESD score (weekly average ≥4 points at baseline and achieves a reduction of HESD score (weekly average) of ≥4 points from baseline at Week 1.

In a further embodiment, the invention relates to the method, use, or the use above, wherein the patient has a baseline HESD score (weekly average) ≥3 points and achieves a reduction of HESD score (weekly average) of ≥3 points from baseline at week 16.

In a further embodiment, the invention relates to the method, use, or the use above, wherein the patient has a baseline HESD score (weekly average) ≥3 points and achieves a reduction of HESD score (weekly average) of ≥3 points from baseline at week 12.

In a further embodiment, the invention relates to the method, use, or the use above, wherein the patient has a baselined HESD score (weekly average ≥3 points at baseline and achieves a reduction of HESD score (weekly average) of ≥3 points from baseline at Week 8.

In a further embodiment, the invention relates to the method, use, or the use above, wherein the patient has a baselined HESD score (weekly average ≥3 points at baseline and achieves a reduction of HESD score (weekly average) of ≥3 points from baseline at Week 4.

In a further embodiment, the invention relates to the method, use, or the use above, wherein the patient has a baselined HESD score (weekly average ≥3 points at baseline and achieves a reduction of HESD score (weekly average) of ≥3 points from baseline at Week 2.

In a further embodiment, the invention relates to the method, use, or the use above, wherein the patient has a baselined HESD score (weekly average ≥3 points at baseline and achieves a reduction of HESD score (weekly average) of ≥3 points from baseline at Week 1.

In a further embodiment, the invention relates to the method, use, or the use above, wherein the patient has a HESD itch score (weekly average) 4 points and achieves a reduction of HEDS itch score (weekly average) of ≥ points from baseline at week 16.

In a further embodiment, the invention relates to the method, use, or the use above, wherein the patient has a baseline HESD itch score (weekly average) ≥4 points and the patient achieves a reduction in HEDS itch score (weekly average) of ≥4 points from baseline at Week 12.

In a further embodiment, the invention relates to the method, use, or the use above, wherein the patient has a baseline HESD itch score (weekly average) ≥4 points and the patient achieves a reduction in HEDS itch score (weekly average) of ≥4 points from baseline at Week 8.

In a further embodiment, the invention relates to the method, use, or the use above, wherein the patient has a baseline HESD itch score (weekly average) ≥4 points and the patient achieves a reduction in HEDS itch score (weekly average) of ≥4 points from baseline at Week 4.

In a further embodiment, the invention relates to the method, use, or the use above, wherein the patient has a baseline HESD itch score (weekly average) ≥4 points and the patient achieves a reduction in HEDS itch score (weekly average) of ≥4 points from baseline at Week 2.

In a further embodiment, the invention relates to the method, use, or the use above, wherein the patient has a baseline HESD itch score (weekly average) ≥4 points and the patient achieves a reduction in HEDS itch score (weekly average) of ≥4 points from baseline at Week 1.

In a further embodiment, the invention relates to the method, use, or the use above, wherein the patient has a baseline HESD itch score (weekly average) ≥3 points and achieves a reduction of HESD itch score (weekly average) of ≥3 points from baseline at Week 16.

In a further embodiment, the invention relates to the method, use, or the use above, wherein the patient has a baseline HESD itch score (weekly average) ≥3 points and achieves a reduction of HESD itch score (weekly average) of ≥3 points from baseline at Week 12.

In a further embodiment, the invention relates to the method, use, or the use above, wherein the patient has a baseline HESD itch score (weekly average) ≥3 points and achieves a reduction of HESD itch score (weekly average) of ≥3 points from baseline at Week 8.

In a further embodiment, the invention relates to the method, use, or the use above, wherein the patient has a baseline HESD itch score (weekly average) ≥3 points and achieves a reduction of HESD itch score (weekly average) of ≥3 points from baseline at Week 4.

In a further embodiment, the invention relates to the method, use, or the use above, wherein the patient has a baseline HESD itch score (weekly average) ≥3 points and achieves a reduction of HESD itch score (weekly average) of ≥3 points from baseline at Week 2.

In a further embodiment, the invention relates to the method, use, or the use above, wherein the patient has a baseline HESD itch score (weekly average) ≥3 points and achieves a reduction of HESD itch score (weekly average) of ≥3 points from baseline at Week 1.

In a further embodiment, the invention relates to the method, use, or the use above, wherein the patient has a baseline HESD itch score (weekly average) ≥3 points and achieves a reduction of HESD itch score (weekly average) of ≥3 points from baseline at 6. Day 5, day 4, day 3, day 2 or within 36 hours or at day 1 or within 24 hours.

In a further embodiment, the invention relates to the method, use, or the use above, wherein the patient has a baseline HESD pain score (weekly average) ≥4 points and achieves a reduction of HESD pain score (weekly average) of ≥4 points from baseline at Week 16.

In a further embodiment, the invention relates to the method, use, or the use above, wherein the patient has a baseline HESD pain score (weekly average) ≥4 points and achieves a reduction of HESD pain score (weekly average) of ≥4 points from baseline at Week 12.

In a further embodiment, the invention relates to the method, use, or the use above, wherein the patient has a baseline HESD pain score (weekly average) ≥4 points and achieves a reduction of HESD pain score (weekly average) of ≥4 points from baseline at Week 8.

In a further embodiment, the invention relates to the method, use, or the use above, wherein the patient has a baseline HESD pain score (weekly average) ≥4 points and achieves a reduction of HESD pain score (weekly average) of ≥4 points from baseline at Week 4.

In a further embodiment, the invention relates to the method, use, or the use above, wherein the patient has a baseline HESD pain score (weekly average) ≥4 points and achieves a reduction of HESD pain score (weekly average) of ≥4 points from baseline at Week 2.

In a further embodiment, the invention relates to the method, use, or the use above, wherein the patient has a baseline HESD pain score (weekly average) ≥4 points and achieves a reduction of HESD pain score (weekly average) of ≥4 points from baseline at Week 1.

In a further embodiment, the invention relates to the method, use, or the use above, wherein the patient has a baseline HESD pain score (weekly average) ≥3 points and achieves a reduction of HESD pain score (weekly average) of ≥3 points from baseline at Week 16.

In a further embodiment, the invention relates to the method, use, or the use above, wherein the patient has a baseline HESD pain score (weekly average) ≥3 points and achieves a reduction of HESD pain score (weekly average) of ≥3 points from baseline at Week 12.

In a further embodiment, the invention relates to the method, use, or the use above, wherein the patient has a baseline HESD pain score (weekly average) ≥3 points and achieves a reduction of HESD pain score (weekly average) of ≥3 points from baseline at Week 8.

In a further embodiment, the invention relates to the method, use, or the use above, wherein the patient has a baseline HESD pain score (weekly average) ≥3 points and achieves a reduction of HESD pain score (weekly average) of ≥3 points from baseline at Week 4.

In a further embodiment, the invention relates to the method, use, or the use above, wherein the patient has a baseline HESD pain score (weekly average) ≥3 points and achieves a reduction of HESD pain score (weekly average) of ≥3 points from baseline at Week 2.

In a further embodiment, the invention relates to the method, use, or the use above, wherein the patient has a baseline HESD pain score (weekly average) ≥3 points and achieves a reduction of HESD pain score (weekly average) of ≥3 points from baseline at Week 1.

In a further embodiment, the invention relates to the method, use, or the use above, wherein the patient has a baseline HESD pain score (weekly average) ≥3 points and achieves a reduction of HESD pain score (weekly average) of ≥3 points from baseline at 6. Day 5, day 4, day 3, day 2 or within 36 hours or at day 1 or within 24 hours.

In a further embodiment, the invention relates to the method, use, or the use above, wherein the patient achieves a reduction of DLQI score of ≥4 points from baseline at Week 16.

In a further embodiment, the invention relates to the method, use, or the use above, wherein the patient achieves a reduction of DLQI score of ≥4 points from baseline at Week 12.

In a further embodiment, the invention relates to the method, use, or the use above, wherein the patient achieves a reduction of DLQI score of ≥4 points from baseline at week 8.

In a further embodiment, the invention relates to the method, use, or the use above, wherein the patient achieves a reduction of DLQI score of ≥4 points from baseline at Week 4.

In a further embodiment, the invention relates to the method, use, or the use above, wherein the patient achieves a reduction of DLQI score of ≥4 points from baseline at Week 2.

In a further embodiment, the invention relates to the method, use, or the use above, wherein the patient achieves a reduction of DLQI score of ≥4 points from baseline at Week 1.

In a further embodiment, the invention relates to the method, use, or the use above, wherein no accumulation of delgocitinib in the body is observed.

In a further embodiment, the invention relates to the method, use, or the use above, wherein the treatment is continued until the patient achieves clear or almost clear skin.

In a further embodiment, the invention relates to the method, use, or the use above, wherein in the event of recurrence of the signs and symptoms of (flares), twice daily treatment of the affected areas is re-initiated as needed.

In a further embodiment, the invention relates to the method, use, or the use above, wherein delgocitinib is dissolved in the aqueous phase of the topical formulation.

In a further embodiment, the invention relates to the method, use, or the use above, wherein the aqueous topical formulation has a pH below about 4.6.

In a further embodiment, the invention relates to the method, use, or the use above, wherein the aqueous topical formulation has a pH between about 3.8 and about 4.6.

In a further embodiment, the invention relates to the method, use, or the use above, wherein the aqueous topical formulation has a pH of about 4.3 or below.

In a further embodiment, the invention relates to the method, use, or the use above, wherein the aqueous topical formulation has a pH of about 4.2 or below.

In a further embodiment, the invention relates to the method, use, or the use above, wherein the aqueous cream comprises a lipid base.

In a further embodiment, the invention relates to the method, use, or the use above, wherein the lipid base is liquid paraffin.

In some embodiments, the acidified aqueous topical composition or aqueous cream is an oil-in water emulsion as described in WO2020/229622.

In some embodiments the acidified topical composition or aqueous cream used according to the invention comprises a large amount of water and is acidified to a pH between 3.8 and 4.6, more preferred a pH around 4.2 using a pharmaceutically acceptable acid such as hydrochloric acid.

In some embodiments the acidified aqueous topical composition or aqueous cream used according to the invention comprises one or more of a base selected from medium chain triglycerides, safflower oil, castor oil, liquid paraffin or mixtures thereof. In some embodiments the base is liquid paraffin.

The base is typically present in an amount from about 50 mg/g to about 500 mg/g, from about 200 mg/g to about 400 mg/g, from about 75 mg/g to about 125 mg/g or in an amount of about 100 mg/g. Suitably the base is liquid paraffin.

In some embodiments the acidified aqueous topical composition or aqueous cream used according to the invention comprises a surfactant, emulsifier or stabilizer and the surfactant, emulsifier or stabilizer is selected from one or more fatty alcohols such as one or more of cetyl alcohol, stearyl alcohol, cetostearyl alcohol, sorbitan esters, sucrose esters, macrogol cetostearyl ether or mixtures thereof. Typically, the surfactant, emulsifier or stabilizer is present in an amount from 40-150 mg/g, 60-120 mg/g or in an amount of about 80-100 mg/g.

Typically, a surfactant, emulsifier or stabilizer may be selected from fatty alcohols, such as cetostearyl alcohol and is present in amounts from about 30 mg/g to about 120 mg/g, e.g. from about 50 mg/g to about 90 mg/g, or in an amount of about 70-75 mg/g. Typically, the surfactant, emulsifier or stabilizer may be selected from cetostearyl alcohol and is present in an amount of about 70-75 mg/g, or in an amount of about 72 mg/g.

In some embodiments the acidified aqueous topical composition or aqueous cream used according to the invention comprises a surfactant, emulsifier or stabilizer and the surfactant, emulsifier or stabilizer is selected from sorbitan esters, sucrose esters, macrogol fatty alcohol ethers or mixtures thereof. For example, the surfactant or emulsifier is macrogol cetostearyl ether.

Typically, the surfactant, emulsifier or stabilizer selected from sorbitan esters, sucrose esters, macrogol cetostearyl ether or mixtures thereof is present at amounts from about 10 mg/g to about 30 mg/g, from about 14 mg/g to about 22 mg/g, or in an amount of about 20-22 mg/g. Typically, a surfactant, emulsifier or stabilizer is macrogol cetostearyl or other fatty alcohol ether in an amount of about 20-22 mg/g or in an amount of about 18 mg/g.

In some embodiments the acidified aqueous topical composition or aqueous cream used according to the invention comprises a buffer or pH regulator. The pharmaceutically acceptable buffer or pH regulator could be one or more of a phosphate or citrate salt, sodium acetate, sodium carbonate, sodium citrate dihydrate, hydrochloric acid or mixtures thereof. For example, the buffer or pH regulator could be one or more of citric acid monohydrate and sodium citrate dihydrate. Typically, a buffer selected from a phosphate, citrate or acetate buffer is used.

The buffer or pH regulator could be present at various amounts from about 0.5 mg/g to about 4 mg/g, or from about 0.7 mg/g to about 2 mg/g, or about 1 g/g.

Typically, the buffer is a citric acid buffer comprising is citric acid monohydrate and sodium citrate dihydrate and the sodium citrate dihydrate present in amounts from about 0.5 mg/g to about 4 mg/g, from about 0.7 mg/g to about 2 mg/g, or in an amount of about 1 g/g and the sodium citrate dihydrate present in amounts from 0 mg/g to about 1 mg/g, from 0 mg/g to about 0.5 mg/g, or is absent.

In some embodiments the acidified aqueous topical composition or aqueous cream used according to the invention comprises a pharmaceutically acceptable preservative such as a preservative selected from benzyl alcohol, sodium dehydroacetate, sorbic acid/salts or mixtures thereof. In one embodiment the preservative is benzyl alcohol.

The preservative could be present at various amounts from about 5 mg/g to about 20 mg/g, from about 7 mg/g to about 13 mg/g, or in an amount of about 10 mg/g. Typically, the preservative is benzyl alcohol and it is present in an from about 5 mg/g to about 20 mg/g, from about 7 mg/g to about 13 mg/g, or in an amount of about 10 mg/g.

In some embodiments the acidified aqueous topical composition or aqueous cream used according to the invention comprises a pharmaceutically acceptable antioxidant such as an antioxidant selected from sodium sulphite, disodium edetate, trisodium edetate, butylhydroxy anisole or mixtures thereof. In one embodiment the antioxidant is butylhydroxy anisole.

The antioxidant could be present at various amounts from about 0.05 mg/g to about 0.3 mg/g, from about 0.1 mg/g to about 0.25 mg/g, or in an amount of about 0.2 mg/g. Typically, the antioxidant is butylhydroxy anisole and is present in and amount from about 0.05 mg/g to about 0.3 mg/g, from about 0.1 mg/g to about 0.25 mg/g, or in an amount of about 0.2 mg/g.

In some embodiments the acidified aqueous topical composition or aqueous cream used according to the invention comprises pharmaceutically acceptable chelating agent such as EDTA, disodium edetate, EGTA, or ethylene diamine. In one embodiment the chelating agent is disodium edetate.

Typically, the chelating agent is present at various amounts from about 0.05 mg/g to about 1.5 mg/g, from about 0.5 mg/g to about 1 mg/g, or in an amount of about 0.6 mg/g. Typically, the chelating agent is EDTA present in and amount from about 0.05 mg/g to about 1.5 mg/g, from about 0.5 mg/g to about 1 mg/g, or in an amount of about 0.6 mg/g.

In some embodiments the acidified aqueous topical composition or aqueous cream used according to the invention comprises a pharmaceutically acceptable acidifying agent could be one or more of a strong acid such as hydrochloric acid or citric acid.

In one embodiment the acidifying agent is hydrochloric acid.

The acidifying agent could be present in an amount from 1 mg/g to about 25 mg/g, from about 10 mg/g to about 20 mg/g, or in an amount of about 17.7 mg/g. Typically, the acidifying agent is hydrochloric acid present in an amount from 1 mg/g to about 25 mg/g, from about 10 mg/g to about 20 mg/g, or in an amount of about 17.7 mg/g.

In some embodiments the acidified aqueous topical composition or aqueous cream used according to the invention contain purified water, which could be present at various amounts from about 500 mg/g to about 900 mg/, and e.g. 760 mg/g.

In some embodiments the acidified aqueous topical composition or aqueous cream used according to the invention comprises 50-500 mg/g of an oily base and 40-150 mg/g of a surfactant, emulsifier or stabilizer.

In some embodiments the acidified aqueous topical composition or aqueous cream used according to the invention comprises 50-500 mg/g of liquid paraffin, 30-120 mg/g of cetostearyl alcohol or another fatty alcohol and 10-30 mg/g of a macrogol cetostearyl ether or other fatty alcohol ether.

In some embodiments the acidified aqueous topical composition or aqueous cream used according to the invention comprises 200-400 mg/g of an oily base and 60-120 mg/g of a surfactant, emulsifier or stabilizer.

In some embodiments the acidified aqueous topical composition or aqueous cream used according to the invention comprises 200-400 mg/g of liquid paraffin, 50-90 mg/g of cetostearyl alcohol or another fatty alcohol and 14-22 mg/g of a macrogol cetostearyl ether or other fatty alcohol ether.

In some embodiments the acidified aqueous topical composition or aqueous cream used according to the invention comprises 75-125 mg/g of an oily base and 80-100 mg/g of a surfactant, emulsifier or stabilizer.

In some embodiments the acidified aqueous topical composition or aqueous cream used according to the invention comprises 75-125 mg/g of liquid paraffin, 70-75 mg/g of cetostearyl alcohol or another fatty alcohol and 14-22 mg/g of a macrogol cetostearyl ether or other fatty alcohol ether.

In some embodiments the acidified aqueous topical composition or aqueous cream used according to the invention comprises 50-500 mg/g of an oily base, 40-150 mg/g of a surfactant, emulsifier or stabilizer and 0.5-4 mg/g of a buffer (total of acid and base).

In some embodiments the acidified aqueous topical composition or aqueous cream used according to the invention comprises 50-500 mg/g of liquid paraffin, 30-120 mg/g of cetostearyl alcohol or another fatty alcohol and 10-30 mg/g of a macrogol cetostearyl ether or other fatty alcohol ether and 0.5-4 mg/g of a buffer (total of base and acid).

In some embodiments the acidified aqueous topical composition or aqueous cream used according to the invention comprises 200-400 mg/g of an oily base, 60-120 mg/g of a surfactant, emulsifier or stabilizer and 0.7-2 mg/g of a of citric acid buffer (total of base and acid) or another suitable buffer.

In some embodiments the acidified aqueous topical composition or aqueous cream used according to the invention comprises 200-400 mg/g of liquid paraffin, 50-90 mg/g of cetostearyl alcohol or another fatty alcohol and 14-22 mg/g of a macrogol cetostearyl ether or other fatty alcohol ether and 0.7-2 mg/g of a of citric acid buffer (total of base and acid) or another suitable buffer.

In some embodiments the acidified aqueous topical composition or aqueous cream used according to the invention comprises 75-125 mg/g of an oily base, 80-100 mg/g of a surfactant, emulsifier or stabilizer and about 1 mg/g of a buffer (total of base and acid).

In some embodiments the acidified aqueous topical composition or aqueous cream used according to the invention comprises 75-125 mg/g of liquid paraffin, 70-75 mg/g of cetostearyl alcohol or another fatty alcohol and 14-22 mg/g of a macrogol cetostearyl ether or other fatty alcohol ether and about 1 mg/g of a of citric acid buffer (total of base and acid) or another suitable buffer.

In some embodiments the acidified aqueous topical composition or aqueous cream used according to the invention comprises 50-500 mg/g of liquid paraffin, 30-120 mg/g of cetostearyl alcohol or another fatty alcohol and 10-30 mg/g of a macrogol cetostearyl ether or other fatty alcohol ether, 0.5-4 mg/g of a buffer (total of base and acid) and 1-25 mg/g of an acidifying agent.

In some embodiments the acidified aqueous topical composition or aqueous cream used according to the invention comprises 200-400 mg/g of an oily base, 60-120 mg/g of a surfactant, emulsifier or stabilizer, 0.7-2 mg/g of a buffer (total of base and acid) or another suitable buffer and 1-25 mg/g of an acidifying agent.

In some embodiments the acidified aqueous topical composition or aqueous cream used according to the invention comprises 200-400 mg/g of liquid paraffin, 50-90 mg/g of cetostearyl alcohol or another fatty alcohol and 14-22 mg/g of a macrogol cetostearyl ether or other fatty alcohol ether and 0.7-2 mg/g of a of citric acid buffer (total of base and acid) or another suitable buffer and 10-20 mg/g of hydrochloric acid or another pharmaceutically acceptable acidifying agent.

In some embodiments the acidified aqueous topical composition or aqueous cream used according to the invention comprises 75-125 mg/g of an oily base, 80-100 mg/g of a surfactant, emulsifier or stabilizer and about 1 mg/g of a buffer (total of base and acid) and 10-20 mg/g of an acidifying agent.

In some embodiments the acidified aqueous topical composition or aqueous cream used according to the invention comprises 75-125 mg/g of liquid paraffin, 70-75 mg/g of cetostearyl alcohol or another fatty alcohol and 14-22 mg/g of a macrogol cetostearyl ether or other fatty alcohol ether and about 1 mg/g of a of citric acid buffer (total of base and acid) or another suitable buffer and 10-20 mg/g of hydrochloric acid or another pharmaceutically acceptable acidifying agent.

According to any of the embodiments above the acidified aqueous topical composition or aqueous cream comprising various amounts of oily base, surfactant, emulsifier or stabilizer, buffer, pH regulator and/or acidifying agent alco contain a preservative, an antioxidant and/or an acidifying agent.

The acidified aqueous topical composition or aqueous cream used according to the invention may be prepared by separately preparing the aqueous and the oil phase and thereafter adding the oil phase to the water phase and mixing it.

### The Water-Phase:

Purified water, pH regulators, buffers, acidifying agents, preservatives, chelating agents were mixed.
Delgocitinib was added and dissolved in the water-phase
pH was adjusted
The water-phase was heated

### The Oil-Phase:

Liquid paraffin, surfactants, emulsifiers, stabilizers, antioxidants were mixed.
The oil-phase was heated
The oil-phase was added to the water-phase and mixed.
The mixture was homogenised cooled. The acidified aqueous topical composition or aqueous cream was subsequently filled into the container closure.

### Exemplary Pharmaceutical Formulations Used According to the Invention:

Component Amount (mg/g): Delgocitinib 20; paraffin liquid 100; cetostearyl alcohol 72; macrogol cetostearyl ether 18; benzyl alcohol 10; citric acid monohydrate 1.0; butylhydroxy anisole 0.2; disodium edetate 0.6; 3M hydrochloric acid 17.7, and purified water 760.

The acidified aqueous topical composition or aqueous cream used according to the invention may be prepared may be prepared as follows:

### The Water-Phase:

Purified water, pH regulators, buffers, acidifying agents, preservatives, chelating agents were mixed.
The drug substance was added and dissolved in the water-phase at a temperature around 15-25° C.
pH was adjusted to 4.0-4.4.
The water-phase was heated to around 65-75° C.

### The Oil-Phase:

Liquid paraffin, surfactants, emulsifiers, stabilizers, antioxidants were mixed.
The oil-phase was heated to around 65-75° C.
The oil-phase was added to the water-phase and mixed.

The mixture was homogenised for approximately 20 min. and subsequently cooled to approximately 30° C. The cream was subsequently filled into the container closure.

### Further Embodiments

1. A method of treating moderate to severe chronic hand eczema in a human patient in need thereof comprising twice daily administration to the skin of said human patient, of an acidified aqueous topical composition comprising comprises 20 mg/g delgocitinib on a free base basis or a pharmaceutically acceptable salt thereof, wherein the patient has disease severity graded as moderate to severe at screening and baseline according to IGA-CHE.

2. The method of embodiment 1, wherein the patient has a HESD itch score (weekly average) of ≥4 points at baseline.

3. The method of any one of embodiments 1-2, wherein the patient: is an adolescent aged 12 to 17 or an adult aged 18 years or above; wherein the patient have hand eczema that has persisted for more than 3 months or returned twice or more within the last 12 months.

4. The method of any one of embodiments 1-3, wherein the patient has a documented recent history of inadequate response to treatment with topical corticosteroids (TCS) or for whom TCS are documented to be otherwise medically inadvisable (e.g. due to important side effects or safety risks).

5. The method of any one of embodiments 1-4, wherein delgocitinib is dissolved in the aqueous phase of the acidified aqueous topical formulation.

6. The method of embodiment 5, wherein the acidified aqueous topical formulation has a pH below about 4.6 or below about 4.4.

7. The method of embodiment 6, wherein the acidified aqueous topical formulation has a pH between about 3.8 and about 4.6.

8. The method of embodiment 7 wherein the aqueous topical formulation has a pH of about 4.3 or below.

9. The method of embodiment 8 wherein the aqueous topical formulation has a pH of about 4.2 or below.

10. The method of any of embodiments 5-9, wherein the aqueous cream comprises a lipid base.

11. The method of embodiment 10, wherein the lipid base is liquid paraffin.

12. The method of any one of embodiments 1-11, wherein the patient achieves an IGA-CHE score of 0 (clear) or 1 (almost clear) with a ≥2-step improvement from baseline at week 16.

13. The method of embodiment 12, wherein the patient achieves an IGA-CHE score of 0 (clear) or 1 (almost clear) with a ≥2-step improvement from baseline at week 8.

14. The method of embodiment 12, wherein the patient achieves an IGA-CHE score of 0 (clear) or 1 (almost clear) with a ≥2-step improvement from baseline at week 4.

15. The method of embodiment 12, wherein the patient achieves an IGA-CHE score of 0 (clear) or 1 (almost clear) with a ≥2-step improvement from baseline at week 2.

16. The method of embodiment 12, wherein the patient achieves an IGA-CHE score of 0 (clear) or 1 (almost clear) with a ≥2-step improvement from baseline at week 1.

17. The method of embodiment 12, wherein the patient achieves an IGA-CHE score of 0 (clear) or 1 (almost clear) with a ≥2-step improvement from baseline at day 5 or 6.

18. The method of embodiment 12, wherein the patient achieves an IGA-CHE score of 0 (clear) or 1 (almost clear) with a ≥2-step improvement from baseline at day 4.

19. The method of embodiment 12, wherein the patient achieves an IGA-CHE score of 0 (clear) or 1 (almost clear) with a ≥2-step improvement from baseline at day 3.

20. The method of embodiment 12, wherein the patient achieves an IGA-CHE score of 0 (clear) or 1 (almost clear) with a ≥2-step improvement from baseline at day 2 or within 36 hours.

21. The method of embodiment 12, wherein the patient achieves an IGA-CHE score of 0 (clear) or 1 (almost clear) with a ≥2-step improvement from baseline at day 1 or within 24 hours.

22. The method of any one of embodiments 1-21, wherein the patient achieves at least 90% improvement in HECSI score from baseline at week 16.

23. The method of embodiment 22, wherein the patient achieves at least 90% improvement in HECSI score from baseline at week 8.

24. The method of embodiment 22, wherein the patient achieves at least 90% improvement in HECSI score from baseline at week 4.

25. The method of embodiment 22, wherein the patient achieves at least 90% improvement in HECSI score from baseline at week 2.

25. The method of embodiment 22, wherein the patient achieves at least 90% improvement in HECSI score from baseline at week 1.
26. The method of embodiment 22, wherein the patient achieves at least 90% improvement in HECSI score from baseline at day 6.
27. The method of embodiment 22, wherein the patient achieves at least 90% improvement in HECSI score from baseline at day 5.
28. The method of embodiment 22, wherein the patient achieves at least 90% improvement in HECSI score from baseline at day 4.
29. The method of embodiment 22, wherein the patient achieves at least 90% improvement in HECSI score from baseline at day 3.
30. The method of embodiment 22, wherein the patient achieves at least 90% improvement in HECSI score from baseline at day 2 or within 36 hours.
31. The method of embodiment 22, wherein the patient achieves at least 90% improvement in HECSI score from baseline at day 1 or within 24 hours.
32. The method of any one of embodiments 1-31, wherein the patient achieves at least 75% improvement in HECSI score from baseline at week 16.
33. The method of embodiment 32, wherein the patient achieves at least 75% improvement in HECSI score from baseline at week 8.
34. The method of embodiment 32, wherein the patient achieves at least 75% improvement in HECSI score from baseline at week 4.
35. The method of embodiment 32, wherein the patient achieves at least 75% improvement in HECSI score from baseline at week 2.
36. The method of embodiment 32, wherein the patient achieves at least 75% improvement in HECSI score from baseline at week 1.
37. The method of embodiment 32, wherein the patient achieves at least 75% improvement in HECSI score from baseline at day 6.
38. The method of embodiment 32, wherein the patient achieves at least 75% improvement in HECSI score from baseline at day 5.
39. The method of embodiment 32, wherein the patient achieves at least 75% improvement in HECSI score from baseline at day 4.
40. The method of embodiment 32, wherein the patient achieves at least 75% improvement in HECSI score from baseline at day 3.
41. The method of embodiment 32, wherein the patient achieves at least 75% improvement in HECSI score from baseline at day 2 or within 36 hours.
42. The method of embodiment 32, wherein the patient achieves at least 75% improvement in HECSI score from baseline at day 1 or within 24 hours.
43. The method of any one of embodiments 1-42, wherein the patient has a baseline HESD score (weekly average) ≥4 points and achieves a reduction of HESD score (weekly average) of ≥4 points from baseline at week 16.
44. The method of embodiment 43, wherein the patient has a baseline HESD score (weekly average) ≥4 points and achieves a reduction of HESD score (weekly average) of ≥4 points from baseline at week 12.
45. The method of embodiment 43, wherein the patient has a baselined HESD score (weekly average ≥4 points at baseline and achieves a reduction of HESD score (weekly average) of ≥4 points from baseline at Week 8.
46. The method of embodiment 43, wherein the patient has a baselined HESD score (weekly average ≥4 points at baseline and achieves a reduction of HESD score (weekly average) of ≥4 points from baseline at Week 4.
47. The method of embodiment 43, wherein the patient has a baselined HESD score (weekly average ≥4 points at baseline and achieves a reduction of HESD score (weekly average) of ≥4 points from baseline at Week 2.
48. The method of embodiment 43, wherein the patient has a baselined HESD score (weekly average ≥4 points at baseline and achieves a reduction of HESD score (weekly average) of ≥4 points from baseline at Week 1.
49. The method of any one of embodiments 1-48, wherein the patient has a baseline HESD itch score (weekly average) 4 points and achieves a reduction of HEDS itch score (weekly average) of 4 points from baseline at week 16.
50. The method of embodiment 49, wherein the patient has a baseline HESD itch score (weekly average) ≥4 points and the patient achieve a reduction in HEDS itch score (weekly average) of ≥4 points from baseline at Week 8.
51. The method of embodiment 49, wherein the patient has a baseline HESD itch score (weekly average) ≥4 points and the patient achieve a reduction in HEDS itch score (weekly average) of ≥4 points from baseline at Week 4.
52. The method of embodiment 49, wherein the patient has a baseline HESD itch score (weekly average) ≥4 points and the patient achieve a reduction in HEDS itch score (weekly average) of ≥4 points from baseline at Week 2.
53. The method of embodiment 49, wherein the patient has a baseline HESD itch score (weekly average) ≥4 points and the patient achieve a reduction in HEDS itch score (weekly average) of ≥4 points from baseline at Week 1.
54. The method of embodiment 49, wherein the patient has a baseline HESD itch score (weekly average) ≥4 points and the patient achieve a reduction in HEDS itch score (weekly average) of ≥4 points from baseline at day 6.
55. The method of embodiment 49, wherein the patient has a baseline HESD itch score (weekly average) ≥4 points and the patient achieve a reduction in HEDS itch score (weekly average) of ≥4 points from baseline at day 5.
56. The method of embodiment 49, wherein the patient has a baseline HESD itch score (weekly average) ≥4 points and the patient achieve a reduction in HEDS itch score (weekly average) of ≥4 points from baseline at day 4.
57. The method of embodiment 49, wherein the patient has a baseline HESD itch score (weekly average) ≥4 points and the patient achieve a reduction in HEDS itch score (weekly average) of ≥4 points from baseline at day 3.
58. The method of embodiment 49, wherein the patient has a baseline HESD itch score (weekly average) ≥4 points and the patient achieve a reduction in HEDS itch score (weekly average) of ≥4 points from baseline at day 2 or within 36 hours.
59. The method of embodiment 49, wherein the patient has a baseline HESD itch score (weekly average) ≥4 points and the patient achieve a reduction in HEDS itch score (weekly average) of ≥4 points from baseline at day 1 or within 24 hours.
60. The method of any one of embodiments 1-59, wherein the patient has a baseline HESD pain score (weekly average) ≥4 points and achieve a reduction of HESD pain score (weekly average) of ≥4 points from baseline at Week 16.
61. The method of embodiment 60, wherein the patient has a baseline HESD pain score (weekly average) ≥4 points and achieves a reduction of HESD pain score (weekly average) of ≥4 points from baseline at Week 8.
62. The method of embodiment 60, wherein the patient has a baseline HESD pain score (weekly average) ≥4 points and achieves a reduction of HESD pain score (weekly average) of ≥4 points from baseline at Week 4.
63. The method of embodiment 60, wherein the patient has a baseline HESD pain score (weekly average) ≥4 points and achieves a reduction of HESD pain score (weekly average) of ≥4 points from baseline at Week 2.
64. The method of embodiment 60, wherein the patient has a baseline HESD pain score (weekly average) ≥4 points and achieves a reduction of HESD pain score (weekly average) of ≥4 points from baseline at Week 1.
65. The method of embodiment 60, wherein the patient has a baseline HESD pain score (weekly average) ≥4 points and the patient achieve a reduction in HEDS pain score (weekly average) of ≥4 points from baseline at day 6.
66. The method of embodiment 60, wherein the patient has a baseline HESD pain score (weekly average) ≥4 points and the patient achieve a reduction in HEDS pain score (weekly average) of ≥4 points from baseline at day 5.
67. The method of embodiment 60, wherein the patient has a baseline HESD pain score (weekly average) ≥4 points and the patient achieve a reduction in HEDS pain score (weekly average) of ≥4 points from baseline at day 4.
68. The method of embodiment 60, wherein the patient has a baseline HESD pain score (weekly average) ≥4 points and the patient achieve a reduction in HEDS pain score (weekly average) of ≥4 points from baseline at day 3.
69. The method of embodiment 60, wherein the patient has a baseline HESD pain score (weekly average) ≥4 points and the patient achieve a reduction in HEDS pain score (weekly average) of ≥4 points from baseline at day 2 or within 36 hours.
70. The method of embodiment 60, wherein the patient has a baseline HESD pain score (weekly average) ≥4 points and the patient achieve a reduction in HEDS pain score (weekly average) of ≥4 points from baseline at day 1 or within 24 hours.
71. The method of any one of claims 1-70, wherein the patient achieves a reduction of DLQI score of ≥4 points from baseline at Week 16.
72. The method of claim 71, wherein the patient achieves a reduction of DLQI score of ≥4 points from baseline at Week 8.
73. The method of claim 71, wherein the patient achieves a reduction of DLQI score of 4 points from baseline at Week 4.
74. The method of claim 71, wherein the patient achieves a reduction of DLQI score of 4 points from baseline at Week 2.
75. The method of claim 71, wherein the patient achieves a reduction of DLQI score of 4 points from baseline at Week 1.
77. The method of any one of embodiments 1-75, wherein the treatment is continued until the patient achieves clear or almost clear skin.
78. The method of embodiment 77, wherein administration is maintained for at least 2 weeks.
79. The method of embodiment 77, wherein administration is maintained for at least 4 weeks.
80. The method of embodiment 77, wherein administration is maintained for at least 8 weeks.
81. The method of embodiment 77, wherein administration is maintained for at least 12 weeks.
82. A method of reducing itch in a human patient with moderate to severe chronic hand eczema, comprising twice daily administration to the skin of said human patient in need thereof, of an acidified aqueous topical composition comprising comprises 20 mg/g delgocitinib on a free base basis or a pharmaceutically acceptable salt thereof, wherein the patient has a baseline HESD itch score (weekly average) 4 points.
83. The method of embodiment 82, wherein the patient: is an adolescent aged 12 to 17 or an adult aged 18 years or above; wherein the patient have hand eczema that has persisted for more than 3 months or returned twice or more within the last 12 months.
84. The method of any one of embodiments 82-83, wherein the patient has a documented recent history of inadequate response to treatment with topical corticosteroids (TCS) or for whom TCS are documented to be otherwise medically inadvisable (e.g. due to important side effects or safety risks).
85. The method of any one of embodiments 82-84, wherein delgocitinib is dissolved in the aqueous phase of the acidified aqueous topical formulation.
86. The method of embodiment 85, wherein the acidified aqueous topical formulation has a pH below about 4.6 or below about 4.4.
87. The method of embodiment 86, wherein the acidified aqueous topical formulation has a pH between about 3.8 and about 4.6.
88. The method of embodiment 87 wherein the aqueous topical formulation has a pH of about 4.3 or below.
89. The method of embodiment 88 wherein the aqueous topical formulation has a pH of about 4.2 or below.
90. The method of any one of embodiments 83-89, wherein the aqueous cream comprises a lipid base.
91. The method of any one of embodiments 82-90, wherein the patient achieves a reduction in HEDS itch score (weekly average) of ≥4 points from baseline at Week 8.
92. The method of embodiment 91, wherein the patient achieves a reduction in HEDS itch score (weekly average) of ≥4 points from baseline at Week 4.
93. The method of embodiment 91, wherein the patient achieves a reduction in HEDS itch score (weekly average) of ≥4 points from baseline at Week 2.
94. The method of embodiment 91, wherein the patient achieves a reduction in HEDS itch score (weekly average) of ≥4 points from baseline at Week 1.
95. The method of embodiment 91, the patient achieves a reduction in HEDS itch score (weekly average) of ≥4 points from baseline at day 6.
96. The method of embodiment 91, wherein the patient achieves a reduction in HEDS itch score (weekly average) of ≥4 points from baseline at day 5.
97. The method of embodiment 91, wherein d the patient achieves a reduction in HEDS itch score (weekly average) of ≥4 points from baseline at day 4.
98. The method of embodiment 91, wherein the patient achieves a reduction in HEDS itch score (weekly average) of ≥4 points from baseline at day 3.
99. The method of embodiment 91, wherein the patient achieves a reduction in HEDS itch score (weekly average) of ≥4 points from baseline at day 2 or within 36 hours.
100. The method of embodiment 91, wherein the patient achieves a reduction in HEDS itch score (weekly average) of ≥4 points from baseline at day 1 or within 24 hours.
101. The method of embodiment 82, wherein the patient achieves a prompt reduction of itch.
102. The method of any one of embodiments 82-101, wherein the patient achieves a statistically significant reduction in HEDS itch score from baseline at day 1 or within 24 hours compared to a patient administered placebo.
103. The method of any one of embodiments 82-101, wherein the patient achieves a 1 point reduction in HEDS itch score from baseline at day 1 or within 24 hours.
104. The method of any one of any one of embodiments 82-101, wherein the patient achieves a statistically significant reduction in HEDS itch score from baseline at day 2 or within 36 hours compared to a patient administered placebo.
105. The method of any one of embodiments 82-101, wherein the patient achieves a 2 point reduction in HEDS itch score from baseline at day 2 or within 36 hours.
106. The method of any one of embodiments 82-101, wherein the patient achieves a 3 point reduction in HEDS itch score from baseline at week 8.
107. The method of any one of embodiments 82-101, wherein the patient achieves a 3 point reduction in HEDS itch score from baseline at week 4.
108. The method of any one of embodiments 82-101, wherein the patient achieves a 3 point reduction in HEDS itch score from baseline at week 2.
109. The method of any one of any one of embodiments 82-101, wherein the patient achieves a 3 point reduction in HEDS itch score from baseline at week 1.
110. The method of embodiments 82-101, wherein the patient achieves a 4 point reduction in HEDS itch score from baseline at week 8.
111. The method of any one of any one of embodiments 82-101, wherein the patient achieves a 4 po of any one of any one int reduction in HEDS itch score from baseline at week 4.
113. The method of any one of embodiments 82-112, wherein the patient achieves significant improvement in IGA CHE score from baseline compared to placebo.
114. The method of any one of embodiment 82-113, wherein the patient achieves an IGA-CHE score of 0 (clear) or 1 (almost clear) with a ≥2-step improvement from baseline at week 8.
115. The method of embodiment 114, wherein the patient achieves an IGA-CHE score of 0 (clear) or 1 (almost clear) with a ≥2-step improvement from baseline at week 4.
116. The method of embodiment 114, wherein the patient achieves an IGA-CHE score of 0 (clear) or 1 (almost clear) with a ≥2-step improvement from baseline at week 2.
117. The method of embodiment 114, wherein the patient achieves an IGA-CHE score of 0 (clear) or 1 (almost clear) with a ≥2-step improvement from baseline at week 1.
118. The method of embodiment 114, wherein the patient achieves an IGA-CHE score of 0 (clear) or 1 (almost clear) with a ≥2-step improvement from baseline at day 5 or 6.
119. The method of embodiment 114, wherein the patient achieves an IGA-CHE score of 0 (clear) or 1 (almost clear) with a ≥2-step improvement from baseline at day 4.
120. The method of embodiment 114, wherein the patient achieves an IGA-CHE score of 0 (clear) or 1 (almost clear) with a ≥2-step improvement from baseline at day 3.
121. The method of embodiment 114, wherein the patient achieves an IGA-CHE score of 0 (clear) or 1 (almost clear) with a ≥2-step improvement from baseline at day 1 or within 24 hours.
122. The method of embodiment 114, wherein the patient achieves an IGA-CHE score of 0 (clear) or 1 (almost clear) with a ≥2-step improvement from baseline at day 2 or within 36 hours.
123. The method of any one of embodiments 82-122, wherein the patient achieves at least 90% improvement in HECSI score from baseline at week 16.
124. The method of embodiment 123, wherein the patient achieves at least 90% improvement in HECSI score from baseline at week 8.
125. The method of embodiment 123, wherein the patient achieves at least 90% improvement in HECSI score from baseline at week 4.
126. The method of embodiment 123, wherein the patient achieves at least 90% improvement in HECSI score from baseline at week 2.
127. The method of embodiment 123, wherein the patient achieves at least 90% improvement in HECSI score from baseline at week 1.
128. The method of embodiment 123, wherein the patient achieves at least 90% improvement in HECSI score from baseline at day 6.
129. The method of embodiment 123, wherein the patient achieves at least 90% improvement in HECSI score from baseline at day 5.
130. The method of embodiment 123, wherein the patient achieves at least 90% improvement in HECSI score from baseline at day 4.
131. The method of embodiment 123, wherein the patient achieves at least 90% improvement in HECSI score from baseline at day 3.
132 The method of embodiment 123, wherein the patient achieves at least 90% improvement in HECSI score from baseline at day 2 or within 36 hours.
133. The method of embodiment 123, wherein the patient achieves at least 90% improvement in HECSI score from baseline at day 1 or within 24 hours.
134. The method of any one of embodiments 82-122, wherein the patient achieves at least 75% improvement in HECSI score from baseline at week 16.
135. The method of embodiment 134, wherein the patient achieves at least 75% improvement in HECSI score from baseline at week 8.
136. The method of embodiment 134, wherein the patient achieves at least 75% improvement in HECSI score from baseline at week 4.
137. The method of embodiment 134, wherein the patient achieves at least 75% improvement in HECSI score from baseline at week 2.
138. The method of embodiment 134, wherein the patient achieves at least 75% improvement in HECSI score from baseline at week 1.
139. The method of embodiment 134, wherein the patient achieves at least 75% improvement in HECSI score from baseline at day 6.
140. The method of embodiment 134, wherein the patient achieves at least 75% improvement in HECSI score from baseline at day 5.
141. The method of embodiment 134, wherein the patient achieves at least 75% improvement in HECSI score from baseline at day 4.
143. The method of embodiment 134, wherein the patient achieves at least 75% improvement in HECSI score from baseline at day 3.
144. The method of embodiment 134, wherein the patient achieves at least 75% improvement in HECSI score from baseline at day 2 or within 36 hours.
145. The method of embodiment 134, wherein the patient achieves at least 75% improvement in HECSI score from baseline at day 1 or within 24 hours.
146. The method of any one of embodiments 82-145, wherein the treatment is continued until the patient achieves clear or almost clear skin.
147. The method of any one of embodiments 82-146, wherein administration is maintained for at least 2 weeks.
148. The method of embodiment 147, wherein administration is maintained for at least 4 weeks.
149. The method of embodiment 147, wherein administration is maintained for at least 8 weeks.
150. The method of embodiment 147, wherein administration is maintained for at least 12 weeks.
151. The method of any one of embodiments 82-150, wherein the patient is not administered other therapeutic agents used to treat chronic hand eczema.
152. The method of any one of embodiments 82-150, wherein the administration of said acidified aqueous formulation does not cause administration site burn.
153. A method of reducing pain in a human patient with moderate to severe chronic hand eczema, comprising twice daily administration to the skin of said human patient in need thereof, of an acidified aqueous topical composition comprising comprises 20 mg/g delgocitinib on a free base basis or a pharmaceutically acceptable salt thereof, wherein the patient has a baseline HESD pain score (weekly average) ≥4 points.
154. The method of embodiment 153, wherein the patient: is an adolescent aged 12 to 17 or an adult aged 18 years or above; wherein the patient have hand eczema that has persisted for more than 3 months or returned twice or more within the last 12 months.
155. The method of any one of embodiments 153-154, wherein the patient has a documented recent history of inadequate response to treatment with topical corticosteroids (TCS) or for whom TCS are documented to be otherwise medically inadvisable (e.g. due to important side effects or safety risks).
156. The method of any one of embodiments 153-155, wherein delgocitinib is dissolved in the aqueous phase of the acidified aqueous topical formulation.
157. The method of embodiment 156, wherein the acidified aqueous topical formulation has a pH below about 4.6 or below about 4.4.
158. The method of embodiment 157, wherein the acidified aqueous topical formulation has a pH between about 3.8 and about 4.6.
159. The method of embodiment 158 wherein the aqueous topical formulation has a pH of about 4.3 or below.
160. The method of embodiment 159 wherein the aqueous topical formulation has a pH of about 4.2 or below.
161. The method of any of embodiments 156-160, wherein the aqueous topical formulation comprises a lipid base.
162. The method of any one of embodiments 153-161, wherein the patient achieves a reduction in HEDS pain score (weekly average) of ≥4 points from baseline at Week 8.
163. The method of embodiment 162, wherein the patient achieves a reduction in HEDS pain score (weekly average) of ≥4 points from baseline at Week 4.
164. The method of embodiment 163, wherein the patient achieves a reduction in HEDS pain score (weekly average) of ≥4 points from baseline at Week 2.
165. The method of embodiment 164, wherein the patient achieves a reduction in HEDS pain score (weekly average) of ≥4 points from baseline at Week 1.
166. The method of embodiment 165, the patient achieves a reduction in HEDS pain score (weekly average) of ≥4 points from baseline at day 6.
167. The method of embodiment 166, wherein the patient achieves a reduction in HEDS pain score (weekly average) of ≥4 points from baseline at day 5.
168. The method of embodiment 167, wherein d the patient achieves a reduction in HEDS pain score (weekly average) of ≥4 points from baseline at day 4.
169. The method of embodiment 168, wherein the patient achieves a reduction in HEDS pain score (weekly average) of ≥4 points from baseline at day 3.
170. The method of embodiment 169, wherein the patient achieves a reduction in HEDS pain score (weekly average) of ≥4 points from baseline at day 2 or within 36 hours.
171. The method of embodiment 170, wherein the patient achieves a reduction in HEDS pain score (weekly average) of ≥4 points from baseline at day 1 or within 24 hours.
172. The method of embodiment 153, wherein the patient achieves a prompt reduction of pain.
173. The method of any one of embodiments 153-172, wherein the patient achieves a statistically significant reduction in HEDS pain score from baseline at day 1 or within 24 hours compared to a patient administered placebo.
174. The method of any one of embodiments 153-172, wherein the patient achieves a 1 point reduction in HEDS pain score from baseline at day 1 or within 24 hours.
175. The method of any one of embodiments 153-172, wherein the patient achieves a statistically significant reduction in HEDS pain score from baseline at day 2 or within 36 hours compared to a patient administered placebo.
176. The method of any one of embodiments 153-172, wherein the patient achieves a 2-point reduction in HEDS pain score from baseline at day 2 or within 36 hours.
177. The method of any one of embodiments 153-172, wherein the patient achieves a 3-point reduction in HEDS pain score from baseline at week 8.
178. The method of any one of embodiments 153-172, wherein the patient achieves a 3-point reduction in HEDS pain score from baseline at week 4.
179. The method of any one of embodiments 153-172, wherein the patient achieves a 3-point reduction in HEDS pain score from baseline at week 2.
180. The method of any one of any one of any one of embodiments 153-172, wherein the patient achieves a 3-point reduction in HEDS pain score from baseline at week 1.
181. The method of any one of any one of any one of any one of embodiments 153-172, wherein the patient achieves a 4-point reduction in HEDS pain score from baseline at week 8.
182. The method of any one of embodiments 153-172, wherein the patient achieves a 4-point reduction in HEDS pain score from baseline at week 4.
183. The method of any one of embodiments 153-182, wherein the patient achieves significant improvement in IGA CHE score from baseline compared to placebo.
184. The method of any one of embodiment 153-182, wherein the patient achieves an IGA-CHE score of 0 (clear) or 1 (almost clear) with a ≥2-step improvement from baseline at week 8.
185. The method of embodiment 184, wherein the patient achieves an IGA-CHE score of 0 (clear) or 1 (almost clear) with a ≥2-step improvement from baseline at week 4.
186. The method of embodiment 184, wherein the patient achieves an IGA-CHE score of 0 (clear) or 1 (almost clear) with a ≥2-step improvement from baseline at week 2.
187. The method of embodiment 184, wherein the patient achieves an IGA-CHE score of 0 (clear) or 1 (almost clear) with a ≥2-step improvement from baseline at week 1.
188. The method of embodiment 184, wherein the patient achieves an IGA-CHE score of 0 (clear) or 1 (almost clear) with a ≥2-step improvement from baseline at day 5 or 6.
189. The method of embodiment 184, wherein the patient achieves an IGA-CHE score of 0 (clear) or 1 (almost clear) with a ≥2-step improvement from baseline at day 4.
190. The method of embodiment 184, wherein the patient achieves an IGA-CHE score of 0 (clear) or 1 (almost clear) with a ≥2-step improvement from baseline at day 3.
191. The method of embodiment 184, wherein the patient achieves an IGA-CHE score of 0 (clear) or 1 (almost clear) with a ≥2-step improvement from baseline at day 1 or within 24 hours.
192. The method of embodiment 184, wherein the patient achieves an IGA-CHE score of 0 (clear) or 1 (almost clear) with a ≥2-step improvement from baseline at day 2 or within 36 hours.
193. The method of any one of embodiments 153-192, wherein the patient achieves at least 90% improvement in HECSI score from baseline at week 16.
194. The method of embodiment 193, wherein the patient achieves at least 90% improvement in HECSI score from baseline at week 8.
195. The method of embodiment 193, wherein the patient achieves at least 90% improvement in HECSI score from baseline at week 4.
196. The method of embodiment 193, wherein the patient achieves at least 90% improvement in HECSI score from baseline at week 2.
197. The method of embodiment 193, wherein the patient achieves at least 90% improvement in HECSI score from baseline at week 1.
198. The method of embodiment 193, wherein the patient achieves at least 90% improvement in HECSI score from baseline at day 6.
199. The method of embodiment 193, wherein the patient achieves at least 90% improvement in HECSI score from baseline at day 5.
200. The method of embodiment 193, wherein the patient achieves at least 90% improvement in HECSI score from baseline at day 4.
201. The method of embodiment 193, wherein the patient achieves at least 90% improvement in HECSI score from baseline at day 3.
202. The method of embodiment 193, wherein the patient achieves at least 90% improvement in HECSI score from baseline at day 2 or within 36 hours.
203. The method of embodiment 193, wherein the patient achieves at least 90% improvement in HECSI score from baseline at day 1 or within 24 hours.
204. The method of any one of embodiments 153-203, wherein the patient achieves at least 75% improvement in HECSI score from baseline at week 16.
205. The method of embodiment 204, wherein the patient achieves at least 75% improvement in HECSI score from baseline at week 8.
206. The method of embodiment 204, wherein the patient achieves at least 75% improvement in HECSI score from baseline at week 4.
207. The method of embodiment 204, wherein the patient achieves at least 75% improvement in HECSI score from baseline at week 2.
208. The method of embodiment 204, wherein the patient achieves at least 75% improvement in HECSI score from baseline at week 1.
209. The method of embodiment 204, wherein the patient achieves at least 75% improvement in HECSI score from baseline at day 6.
210. The method of embodiment 204, wherein the patient achieves at least 75% improvement in HECSI score from baseline at day 5.
211. The method of embodiment 204, wherein the patient achieves at least 75% improvement in HECSI score from baseline at day 4.
213. The method of embodiment 204, wherein the patient achieves at least 75% improvement in HECSI score from baseline at day 3.
214. The method of embodiment 204, wherein the patient achieves at least 75% improvement in HECSI score from baseline at day 2 or within 36 hours.
215. The method of embodiment 204, wherein the patient achieves at least 75% improvement in HECSI score from baseline at day 1 or within 24 hours.
216. The method of any one of embodiments 153-215, wherein the treatment is continued until the patient achieves clear or almost clear skin.
217. The method of any one of embodiments 82-146, wherein administration is maintained for at least 2 weeks.
218. The method of embodiment 217, wherein administration is maintained for at least 4 weeks.
219. The method of embodiment 217, wherein administration is maintained for at least 8 weeks.
220. The method of embodiment 217, wherein administration is maintained for at least 12 weeks.
221. The method of any one of embodiments 153-220, wherein the patient is not administered other therapeutic agents used to treat chronic hand eczema.
222. The method of any one of embodiments 153-220, wherein the administration of said acidified aqueous formulation does not cause administration site burn.
223. A method of treating moderate to severe chronic hand eczema in a human patient comprising twice daily administration to the skin of said human patient in need thereof, of an acidified aqueous topical composition comprising comprises 20 mg/g delgocitinib on a free base basis or a pharmaceutically acceptable salt thereof, wherein said administration is maintained for at least 12 weeks, wherein the patient achieves achieves an IGA-CHE score of 0 (clear) or 1 (almost clear) with a ≥2-step improvement from baseline at week 12, and wherein the patient has a baseline HESD itch score (weekly average) ≥4 points and the patient achieves a reduction in HEDS itch score (weekly average) of ≥4 points from baseline at Week 12.
224. The method of embodiment 223, wherein the patient is adult aged 18 years or above, and wherein the patient have hand eczema that has persisted for more than 3 months or returned twice or more within the last 12 months.
225. A method of treating moderate to severe chronic hand eczema in a human patient comprising twice daily administration to the skin of said human patient in need thereof, of an acidified aqueous topical composition comprising comprises 20 mg/g delgocitinib on a free base basis or a pharmaceutically acceptable salt thereof, wherein said administration is maintained for at least 8 weeks, wherein the patient achieves an IGA-CHE score of 0 (clear) or 1 (almost clear) with a ≥2-step improvement from baseline at week 8, and wherein the patient has a baseline HESD itch score (weekly average) ≥4 points and the patient achieves a reduction in HEDS itch score (weekly average) of ≥4 points from baseline at Week 8.
226. The method of embodiment 224, wherein the patient is adult aged 18 years or above, and wherein the patient has hand eczema that has persisted for more than 3 months or returned twice or more within the last 12 months.
227. A method of treating moderate to severe chronic hand eczema in a human patient comprising twice daily administration to the skin of said human patient in need thereof, of an acidified aqueous topical composition comprising comprises 20 mg/g delgocitinib on a free base basis or a pharmaceutically acceptable salt thereof, wherein said administration is maintained for at least 6 weeks, wherein the patient achieves acan IGA-CHE score of 0 (clear) or 1 (almost clear) with a ≥2-step improvement from baseline at week 6, and wherein the patient has a baseline HESD itch score (weekly average) ≥4 points and the patient achieves a reduction in HEDS itch score (weekly average) of ≥4 points from baseline at Week 6.
228. The method of embodiment 224, wherein the patient is adult aged 18 years or above, and wherein the patient has hand eczema that has persisted for more than 3 months or returned twice or more within the last 12 months.
229. A method of treating moderate chronic hand Eczema in a human patient comprising twice daily administration to the skin of said human patient in need thereof, of a topical formulation n comprising comprises 20 mg/g delgocitinib on a free base basis or a pharmaceutically acceptable salt thereof.
230. A method of treating severe chronic hand Eczema in a human patient comprising twice daily administration to the skin of said human patient in need thereof, of a topical formulation n comprising comprises 20 mg/g delgocitinib on a free base basis or a pharmaceutically acceptable salt thereof.
231. The method of any one of embodiments 229-230, wherein the patient has a baseline HESD itch score (weekly average) ≥4 points.
232. The method according to any one of embodiments 229-231, wherein the patient has an IGA-CHE score of 3 or 4.
233. The method of any one of embodiments 229-232, wherein the patient has a documented recent history of inadequate response to treatment with topical corticosteroids (TCS) or for whom TCS are documented to be otherwise medically inadvisable (e.g. due to important side effects or safety risks).
234. The method of embodiment 233, wherein the patient has a documented recent history of inadequate response to treatment with topical corticosteroids (TCS).
235. The method of embodiments 233, wherein topical corticosteroids (TCS) is documented to be medically inadvisable for on in patient.
236. The method of any one of any one of embodiments 229-235, wherein the patient is an adult of 18 years or above.
237. The method of embodiments 229-235, wherein the patient is an adolescent of 12-17 years.
238. The method of any one of embodiments 229-237, where in the topical formulation is administered for at least 2 weeks.
239. The method of embodiment 238, where in the topical formulation is administered for at least 4 weeks.
240. The method of embodiment 239, where in the topical formulation is administered for at least 8 weeks.
241. The method of embodiment 240, where in the topical formulation is administered for at least 12 weeks.
242. The method of embodiment 241, where in the topical formulation is administered for at least 16 weeks or more.
243. The method of any one of embodiments 229-242, wherein the patient achieves an IGA-CHE score of 0 (clear) or 1 (almost clear) with a ≥2-step improvement from baseline at week 8.
244. The method of any one of embodiments 229-242, wherein the patient achieves an IGA-CHE score of 0 (clear) or 1 (almost clear) with a ≥2-step improvement from baseline at week 6.
245. The method of any one of embodiments 229-242, wherein the patient achieves an IGA-CHE score of 0 (clear) or 1 (almost clear) with a ≥2-step improvement from baseline at week 4.
246. The method of any one of embodiments 229-242, wherein the patient achieves an IGA-CHE score of 0 (clear) or 1 (almost clear) with a ≥2-step improvement from baseline at week 2.
247. The method of any one of embodiments 229-242, wherein the patient has a baseline HESD itch score (weekly average) ≥4 points and the patient achieves a reduction in HEDS itch score (weekly average) of ≥4 points from baseline at Week 8 and wherein the patient achieves an IGA-CHE score of 0 (clear) or 1 (almost clear) with a ≥2-step improvement from baseline at week 8.
248. The method of any one of embodiments 229-242, wherein the patient has a baseline HESD itch score (weekly average) ≥4 points and the patient achieves a reduction in HEDS itch score (weekly average) of ≥4 points from baseline at Week 6 and wherein the patient achieves an IGA-CHE score of 0 (clear) or 1 (almost clear) with a ≥2-step improvement from baseline at week 6.
249. The method of any one of embodiments 229-242, wherein the patient has a baseline HESD itch score (weekly average) ≥4 points and wherein the patient achieves a reduction in HEDS itch score (weekly average) of ≥4 points from baseline at Week 4 and wherein the patient achieves an IGA-CHE score of 0 (clear) or 1 (almost clear) with a ≥2-step improvement from baseline at week 4.
250. The method of any one of embodiments 229-242, wherein the patient has a baseline HESD itch score (weekly average) ≥4 points and the patient achieves a reduction in HEDS itch score (weekly average) of ≥4 points from baseline at Week 2 and wherein the patient achieves an IGA-CHE score of 0 (clear) or 1 (almost clear) with a ≥2-step improvement from baseline at week 2.
251. The method of any one of embodiments 229-242, wherein the patient has a baseline HESD itch score (weekly average) ≥4 points and the patient achieves a reduction in HEDS itch score (weekly average) of ≥4 points from baseline at Week 1 and wherein the patient achieves an IGA-CHE score of 0 (clear) or 1 (almost clear) with a ≥2-step improvement from baseline at week 1.
252. The method of any one of embodiments 229-242, wherein the patient has a baseline HESD pain score (weekly average) ≥4 points and the patient achieves a reduction in HEDS pain score (weekly average) of ≥4 points from baseline at Week 8 and wherein the patient achieves an IGA-CHE score of 0 (clear) or 1 (almost clear) with a ≥2-step improvement from baseline at week 8.
253. The method of any one of embodiments 229-242, wherein the patient has a baseline HESD pain score (weekly average) ≥4 points and the patient achieves a reduction in HEDS pain score (weekly average) of ≥4 points from baseline at Week 6 and wherein the patient achieves an IGA-CHE score of 0 (clear) or 1 (almost clear) with a ≥2-step improvement from baseline at week 6.
254. The method of any one of embodiments 229-242, wherein the patient has a baseline HESD pain score (weekly average) ≥4 points and wherein the patient achieves a reduction in HEDS pain score (weekly average) of ≥4 points from baseline at Week 4 and wherein the patient achieves an IGA-CHE score of 0 (clear) or 1 (almost clear) with a ≥2-step improvement from baseline at week 4.
255. The method of any one of embodiments 229-242, wherein the patient has a baseline HESD pain score (weekly average) ≥4 points and the patient achieves a reduction in HEDS pain score (weekly average) of ≥4 points from baseline at Week 2 and wherein the patient achieves an IGA-CHE score of 0 (clear) or 1 (almost clear) with a ≥2-step improvement from baseline at week 2.
256. The method of any one of embodiments 229-242, wherein the patient has a baseline HESD pain score (weekly average) ≥4 points and the patient achieves a reduction in HEDS pain score (weekly average) of ≥4 points from baseline at Week 1 and wherein the patient achieves an IGA-CHE score of 0 (clear) or 1 (almost clear) with a ≥2-step improvement from baseline at week 1.
257. The method of any one of embodiments 229-242, wherein the patient achieves an IGA-CHE score of 0 (clear) or 1 (almost clear) with a ≥2-step improvement from baseline at week 8 and at last 90% improvement in HECSI score from baseline at week 8.
258. The method of any one of embodiments 229-242, wherein the patient achieves an IGA-CHE score of 0 (clear) or 1 (almost clear) with a ≥2-step improvement from baseline at week 6 and at last 90% improvement in HECSI score from baseline at week 6.
259. The method of any one of embodiments 229-242, wherein the patient achieves an IGA-CHE score of 0 (clear) or 1 (almost clear) with a ≥2-step improvement from baseline at week 4 and at last 90% improvement in HECSI score from baseline at week 4.
260. The method of any one of embodiments 229-242, wherein the patient achieves an IGA-CHE score of 0 (clear) or 1 (almost clear) with a ≥2-step improvement from baseline at week 2 and at last 90% improvement in HECSI score from baseline at week 2.
261. The method of any one of embodiments 229-242, wherein the patient achieves an IGA-CHE score of 0 (clear) or 1 (almost clear) with a ≥2-step improvement from baseline at week 1 and at last 90% improvement in HECSI score from baseline at week 1.
262. The method of any one of embodiments 229-242, wherein the patient achieves an IGA-CHE score of 0 (clear) or 1 (almost clear) with a ≥2-step improvement from baseline at week 8 and at last 75% improvement in HECSI score from baseline at week 8.
263. The method of any one of embodiments 229-242, wherein the patient achieves an IGA-CHE score of 0 (clear) or 1 (almost clear) with a ≥2-step improvement from baseline at week 6 and at last 90% improvement in HECSI score from baseline at week 6.
264. The method of any one of embodiments 229-242, wherein the patient achieves an IGA-CHE score of 0 (clear) or 1 (almost clear) with a ≥2-step improvement from baseline at week 4 and at last 75% improvement in HECSI score from baseline at week 4.
265. The method of any one of embodiments 229-242, wherein the patient achieves an IGA-CHE score of 0 (clear) or 1 (almost clear) with a ≥2-step improvement from baseline at week 2 and at last 75% improvement in HECSI score from baseline at week 2.
266. The method of any one of embodiments 229-242, wherein the patient achieves an IGA-CHE score of 0 (clear) or 1 (almost clear) with a ≥2-step improvement from baseline at week 1 and at last 75% improvement in HECSI score from baseline at week 1.
267. The method of any one of embodiments 229-242, wherein the patient has a baseline HESD itch score (weekly average) ≥4 points and the patient achieves a reduction in HEDS itch score (weekly average) of ≥4 points from baseline, an IGA-CHE score of 0 (clear) or 1 (almost clear) with a ≥2-step improvement from baseline and at last 75% improvement in HECSI score from baseline at week 8.
268. The method of any one of embodiments 229-242, wherein the patient has a baseline HESD itch score (weekly average) ≥4 points and the patient achieves a reduction in HEDS itch score (weekly average) of ≥4 points from baseline, an IGA-CHE score of 0 (clear) or 1 (almost clear) with a ≥2-step improvement from baseline and at last 75% improvement in HECSI score from baseline at week 6.
269. The method of any one of embodiments 229-242, wherein the patient has a baseline HESD itch score (weekly average) ≥4 points and the patient achieves a reduction in HEDS itch score (weekly average) of ≥4 points from baseline, an IGA-CHE score of 0 (clear) or 1 (almost clear) with a ≥2-step improvement from baseline and at last 75% improvement in HECSI score from baseline at week 4.
270. The method of any one of embodiments 229-242, wherein the patient has a baseline HESD itch score (weekly average) ≥4 points and the patient achieves a reduction in HEDS itch score (weekly average) of ≥4 points from baseline, an IGA-CHE score of 0 (clear) or 1 (almost clear) with a ≥2-step improvement from baseline and at last 75% improvement in HECSI score from baseline at week 2.
271. The method of any one of embodiments 229-242, wherein the patient has a baseline HESD itch score (weekly average) ≥4 points and the patient achieves a reduction in HEDS itch score (weekly average) of ≥4 points from baseline, an IGA-CHE score of 0 (clear) or 1 (almost clear) with a ≥2-step improvement from baseline and at last 75% improvement in HECSI score from baseline at week 1.
272. The method of any one of embodiments 229-242, wherein the patient has a baseline HESD itch score (weekly average) ≥4 points and the patient achieves a reduction in HEDS itch score (weekly average) of ≥4 points from baseline, an IGA-CHE score of 0 (clear) or 1 (almost clear) with a ≥2-step improvement from baseline at week 8 and at last 90% improvement in HECSI score from baseline at week 8.
273. The method of any one of embodiments 229-242, wherein the patient has a baseline HESD itch score (weekly average) ≥4 points and the patient achieves a reduction in HEDS itch score (weekly average) of ≥4 points from baseline, an IGA-CHE score of 0 (clear) or 1 (almost clear) with a ≥2-step improvement from baseline at week 8 and at last 90% improvement in HECSI score from baseline at week 6.
274. The method of any one of embodiments 229-242, wherein the patient has a baseline HESD itch score (weekly average) ≥4 points and the patient achieves a reduction in HEDS itch score (weekly average) of ≥4 points from baseline, an IGA-CHE score of 0 (clear) or 1 (almost clear) with a ≥2-step improvement from baseline at week 8 and at last 90% improvement in HECSI score from baseline at week 4.
275. The method of any one of embodiments 229-242, wherein the patient has a baseline HESD itch score (weekly average) ≥4 points and the patient achieves a reduction in HEDS itch score (weekly average) of ≥4 points from baseline, an IGA-CHE score of 0 (clear) or 1 (almost clear) with a ≥2-step improvement from baseline at week 8 and at last 90% improvement in HECSI score from baseline at week 2.
276. The method of any one of embodiments 229-242, wherein the patient has a baseline HESD itch score (weekly average) ≥4 points and the patient achieves a reduction in HEDS itch score (weekly average) of ≥4 points from baseline, an IGA-CHE score of 0 (clear) or 1 (almost clear) with a ≥2-step improvement from baseline at week 8 and at last 90% improvement in HECSI score from baseline at week 1.
277. The method of any one of embodiments 229-242, wherein the patient has a baseline HESD pain score (weekly average) ≥4 points and the patient achieves a reduction in HEDS pain score (weekly average) of ≥4 points from baseline, an IGA-CHE score of 0 (clear) or 1 (almost clear) with a ≥2-step improvement from baseline and at last 75% improvement in HECSI score from baseline at week 8.
278. The method of any one of embodiments 229-242, wherein the patient has a baseline HESD pain score (weekly average) ≥4 points and the patient achieves a reduction in HEDS pain score (weekly average) of ≥4 points from baseline, an IGA-CHE score of 0 (clear) or 1 (almost clear) with a ≥2-step improvement from baseline and at last 75% improvement in HECSI score from baseline at week 6.
279. The method of any one of embodiments 229-242, wherein the patient has a baseline HESD pain score (weekly average) ≥4 points and the patient achieves a reduction in HEDS pain score (weekly average) of ≥4 points from baseline, an IGA-CHE score of 0 (clear) or 1 (almost clear) with a ≥2-step improvement from baseline and at last 75% improvement in HECSI score from baseline at week 4.
280. The method of any one of embodiments 229-242, wherein the patient has a baseline HESD pain score (weekly average) ≥4 points and the patient achieves a reduction in HEDS pain score (weekly average) of ≥4 points from baseline, an IGA-CHE score of 0 (clear) or 1 (almost clear) with a ≥2-step improvement from baseline and at last 75% improvement in HECSI score from baseline at week 2.
281. The method of any one of embodiments 229-242, wherein the patient has a baseline HESD pain score (weekly average) ≥4 points and the patient achieves a reduction in HEDS pain score (weekly average) of ≥4 points from baseline, an IGA-CHE score of 0 (clear) or 1 (almost clear) with a ≥2-step improvement from baseline and at last 75% improvement in HECSI score from baseline at week 1.
282. The method of any one of embodiments 229-242, wherein the patient has a baseline HESD pain score (weekly average) ≥4 points and the patient achieves a reduction in HEDS pain score (weekly average) of ≥4 points from baseline, an IGA-CHE score of 0 (clear) or 1 (almost clear) with a ≥2-step improvement from baseline at week 8 and at last 90% improvement in HECSI score from baseline at week 8.
283. The method of any one of embodiments 229-242, wherein the patient has a baseline HESD pain score (weekly average) ≥4 points and the patient achieves a reduction in HEDS pain score (weekly average) of ≥4 points from baseline, an IGA-CHE score of 0 (clear) or 1 (almost clear) with a ≥2-step improvement from baseline at week 8 and at last 90% improvement in HECSI score from baseline at week 6.
284. The method of any one of embodiments 229-242, wherein the patient has a baseline HESD pain score (weekly average) ≥4 points and the patient achieves a reduction in HEDS pain score (weekly average) of ≥4 points from baseline, an IGA-CHE score of 0 (clear) or 1 (almost clear) with a ≥2-step improvement from baseline at week 8 and at last 90% improvement in HECSI score from baseline at week 4.
285. The method of any one of embodiments 229-242, wherein the patient has a baseline HESD pain score (weekly average) ≥4 points and the patient achieves a reduction in HEDS pain score (weekly average) of ≥4 points from baseline, an IGA-CHE score of 0 (clear) or 1 (almost clear) with a ≥2-step improvement from baseline at week 8 and at last 90% improvement in HECSI score from baseline at week 2.
286. The method of any one of embodiments 229-242, wherein the patient has a baseline HESD pain score (weekly average) ≥4 points and the patient achieves a reduction in HEDS pain score (weekly average) of ≥4 points from baseline, an IGA-CHE score of 0 (clear) or 1 (almost clear) with a ≥2-step improvement from baseline at week 8 and at last 90% improvement in HECSI score from baseline at week 1.
287. The method of any one of embodiments 229-286, wherein the patient: is an adolescent aged 12 to 17 or an adult aged 18 years or above; wherein the patient have hand eczema that has persisted for more than 3 months or returned twice or more within the last 12 months.
288. The method of any one of embodiments 229-286, wherein the patient has a documented recent history of inadequate response to treatment with topical corticosteroids (TCS) or for whom TCS are documented to be otherwise medically inadvisable (e.g. due to important side effects or safety risks).
289. The method of any one of embodiments 229-286, wherein the topical formulation is an aqueous cream.
290. The method of embodiment 289, wherein the delgocitinib is dissolved in the aqueous phase of the aqueous cream.
291. The method of embodiment 290, wherein the aqueous cream is acidified and has a pH below about 4.6.
292. The method of embodiment 291 wherein the aqueous cream has a pH between about 3.8 and about 4.6.
293. The method of embodiment 292 wherein the aqueous topical formulation has a pH of about 4.3 or below.
294. The method of embodiment 293 wherein the aqueous topical formulation has a pH of about 4.2 or below.
295. The method of any one of embodiments 291-294, wherein the aqueous cream comprises a lipid base.
296. The method of embodiment 295, wherein the lipid base is liquid paraffin.
297. The method according to any one of embodiments 1-296, wherein the acidified aqueous topical composition or aqueous cream comprises surfactants, emulsifiers, or stabilizers.
298. The method according to claims 297, wherein the surfactants, emulsifiers, or stabilizers is selected from one or more of cetostearyl alcohol and macrogol cetostearyl ether.
299. The method of embodiment 298, wherein the surfactants, emulsifiers, or stabilizers is selected from at least about 30 mg/g to about 80 mg/g cetostearyl alcohol.
300. The method according to anyone of embodiment 298-299, the surfactants, emulsifiers, or stabilizers is selected from at least about 12 mg/g to about 22 mg/g macrogol cetosteary ether.
301. The method according to embodiment 296, wherein the aqueous cream or acidified aqueous topical composition or comprises liquid paraffin present in an amount from about 60 mg/g to about 140 mg/g, cetostearyl alcohol in an amount from about 60 mg/g to about 90 mg/g and macrogol cetostearyl ether present in an amount from about 14 mg/g to about 22 mg/g.
302. The method according to embodiment 301, wherein the aqueous cream or acidified aqueous topical composition or comprises liquid paraffin in an amount of about 100 mg/g, the cetostearyl alcohol in an amount of about 72 mg/g, and macrogol cetostearyl ether in an amount of about 18 mg/g.
303. The method according to anyone of embodiments 296-302, wherein the aqueous cream or acidified aqueous topical composition or comprises a buffer or pH regulator.
304. The method of embodiment 303, wherein the buffer is a phosphate, a citrate or acetate buffer.
305. The method of embodiment 304, wherein the aqueous cream or acidified aqueous topical composition comprises citric acid monohydrate in an amount of from about 0.5 mg/g to about 4 mg/g.
306. The method according to embodiment 305, wherein the aqueous cream or acidified aqueous topical composition comprises sodium citrate dihydrate in an amount of from 0 mg/g to about 1 mg/g.
307. The method according to any one of embodiments 296-306, wherein the aqueous cream or acidified aqueous topical composition comprises benzyl alcohol in an amount from about 7 mg/g to about 13 mg/g.
308. The method according to embodiment 307, wherein benzyl alcohol in an amount from about 9 mg/g to about 11 mg/g.
309. The method according to any one of embodiments 296-308, wherein the aqueous cream or acidified aqueous topical composition comprises butylhydroxy anisole is in an amount of from about 0.05 mg/g to about 0.3 mg/g.
310. The method according to embodiment 309, wherein the aqueous cream or acidified aqueous topical composition comprises disodium edetate in an amount from about 0.05 mg/g to about 1.5 mg/g.
311. The method according to any one of embodiments 296-308, wherein the aqueous cream or acidified aqueous topical composition comprises hydrochloric acid in an amount from 0.1 mg/g to about 25 mg/g.
312. The method according to embodiment 311, wherein the aqueous cream or acidified aqueous topical composition comprises hydrochloric acid in an amount 10 mg/g to about 20 mg/g.
313. The method according to any one of embodiments 296-308, wherein the aqueous cream or acidified aqueous topical composition comprises comprises purified water is present in an amount from about 500 mg/g to about 900 mg/g.
314. The method according to any one of embodiments 296-313, wherein the aqueous cream or acidified aqueous topical composition comprises delgocitinib 20 mg/g; liquid paraffin, cetostearyl alcohol, macrogol cetostearyl ether, benzyl alcohol, citric acid, butylhydroxy anisole, disodium edetate, hydrochloric and purified water.
315. The method according to embodiment 314, wherein the aqueous cream or acidified aqueous topical composition comprises delgocitinib 20 mg/g; liquid paraffin, 100 mg/g; cetostearyl alcohol, 72 mg/g; macrogol cetostearyl ether, 18 mg/g; benzyl alcohol, 10 mg/g; citric acid monohydrate, 1 mg/g; butylhydroxy anisole, 0.2 mg/g; disodium edetate, 0.6 mg/g; 3M hydrochloric acid, 17.7 mg/g; and purified water, 760 mg/g.
316. The method according to any one of embodiments 296-315, wherein the skin permeability of the aqueous cream or acidified aqueous topical composition is about the same or improved compared to a 30 mg ointment comprising 30 mg/g delgocitinib, 820 mg/g white soft paraffin, 50 mg/g hard paraffin and 100 mg/g Squalene as measured in open flow microperfusion testing in pigs.
317. The method of any one of embodiments 1-316, wherein in the event of recurrence of the signs and symptoms of (flares), twice daily treatment of the affected areas is re-initiated as needed.
318. The method of any one of embodiments 1-317, wherein the administration to the skin of a human patient is and administration to the hands and wrists of a human patient.
319. An acidified aqueous topical composition comprising comprises 20 mg/g delgocitinib on a free base basis or a pharmaceutically acceptable salt thereof according to any one of the embodiments 1-318 for use in a method according to any one of embodiments 1-318.
320. The use of delgocitinib for the manufacture of an acidified aqueous topical composition comprising comprises 20 mg/g delgocitinib on a free base basis or a pharmaceutically acceptable salt thereof according to any one of embodiments 1-318 for use in a method according to anyone of claims 1-318.

### Example 1

Delta 1 and Delta 2, two phase 3 clinicals trial to confirm efficacy and evaluate safety of twice-daily delgocitinib cream 20 mg/g compared with cream vehicle for a 16-week treatment period in adult subjects with moderate to severe chronic hand eczema (Delta 1).

The primary objective primary endpoint was IGA-CHE TS at Week 16.

The primary objective secondary endpoint was HECSI-75 at Week 16, HECSI-75 at Week 8, HECSI-90 at Week 16, IGA-CHE TS at Week 8, IGA-CHE TS at Week 4 and percentage change in HECSI score from baseline to Week 16.

Other endpoints are disclosed in table 7 and 8 above.

For DELTA 1, 487 patients were randomized to delgocitinib cream (n=325; 66.7%) and cream vehicle (n=162; 33.3%) from 6 countries (Canada [n=97], France [n=81], Germany [n=135], Italy [n=45], Poland [n=105], and United Kingdom [n=24]). For DELTA 2, 473 patients were randomized from 7 countries (Belgium [n=22], Canada [n=97], Denmark [n=22], Germany [n=147], Netherlands [n=24], Poland [n=96], and Spain [n=65]), with 314 (66.4%) receiving delgocitinib cream and 159 (33.6%) cream vehicle. Overall, 487 (DELTA 1) and 472 (DELTA 2) patients were included in the full analysis set and safety analysis set. In total, 459 (94.3%) and 420 (88.8%) patients completed DELTA 1 and DELTA 2, respectively. Overall, 6.2% (DELTA 1) and 7.0% (DELTA 2) of patients treated with delgocitinib cream, and 13.0% (DELTA 1) and 23.3% (DELTA 2) of those in the cream vehicle groups discontinued study treatment. For both studies, baseline demographics and patient characteristics were comparable between patients treated with delgocitinib cream and patients treated with cream vehicle (Table 11). Overall, 99.4% (DELTA 1) and 98.5% (DELTA 1) of patients had a recent history of inadequate response to TCS treatment (Table 12).

### Baseline Demographics and Characteristics:

TABLE 11
Patient demographics and baseline characteristics in DELTA 1 and DELTA 2

| | DELTA 1 | | | | | | DELTA 2 | | | | | |
|---|---|---|---|---|---|---|---|---|---|---|---|---|
| | | | Delgocitinib | | Cream | | | | Delgocitinib | | Cream | |
| | Total | | cream 20 mg/g | | vehicle | | Total | | cream 20 mg/g | | vehicle | |
| | (N = 487) | | (N = 325) | | (N = 162) | | (N = 473) | | (N = 314) | | (N = 159) | |
| Age, median years | 44.0 | (19-87) | 45.0 | (19-87) | 42.5 | (20-73) | 44.0 | (18-86) | 46.0 | (18-83) | 42.0 | (18-86) |
| (min-max) | | | | | | | | | | | | |
| Sex, n (%) | | | | | | | | | | | | |
| Male | 181 | (37.2) | 123 | (37.8) | 58 | (35.8) | 161 | (34.0) | 110 | (35.0) | 51 | (32.1) |
| Female | 306 | (62.8) | 202 | (62.2) | 104 | (64.2) | 312 | (66.0) | 204 | (65.0) | 108 | (67.9) |
| Race, n (%) | | | | | | | | | | | | |
| White | 427 | (87.7) | 283 | (87.1) | 144 | (88.9) | 441 | (93.2) | 295 | (93.9) | 146 | (91.8) |
| Black or African | 4 | (0.8) | 3 | (0.9) | 1 | (0.6) | 3 | (0.6) | 2 | (0.6) | 1 | (0.6) |
| American | | | | | | | | | | | | |
| Asian | 19 | (3.9) | 14 | (4.3) | 5 | (3.1) | 15 | (3.2) | 8 | (2.5) | 7 | (4.4) |
| Other/Not reported | 37 | (7.6) | 25 | (7.7) | 12 | (7.4) | 14 | (3.0) | 9 | (2.9) | 5 | (3.1) |
| Region, n (%) | | | | | | | | | | | | |
| Europe | 390 | (80.1) | 260 | (80.0) | 130 | (80.2) | 376 | (79.5) | 250 | (79.6) | 126 | (79.2) |
| North America | 97 | (19.9) | 65 | (20.0) | 32 | (19.8) | 97 | (20.5) | 64 | (20.4) | 33 | (20.8) |
| Age at onset of CHE, | 32.0 | (0-87) | 33.0 | (0-87) | 30.0 | (0-72) | 34.0 | (0-83) | 35.0 | (0-83) | 32.0 | (0-77) |
| median years (min- | | | | | | | | | | | | |
| max) | | | | | | | | | | | | |
| Duration of CHE, | 6.0 | (0-61) | 6.0 | (0-61) | 5.5 | (0-53) | 5.0 | (0-59) | 4.0 | (0-59) | 5.0 | (0-52) |
| median years (min- | | | | | | | | | | | | |
| max) | | | | | | | | | | | | |
| IGA-CHE, n (%) | | | | | | | | | | | | |
| Moderate | 327 | (67.1) | 218 | (67.1) | 109 | (67.3) | 360 | (76.1) | 239 | (76.1) | 121 | (76.1) |
| Severe | 160 | (32.9) | 107 | (32.9) | 53 | (32.7) | 113 | (23.9) | 75 | (23.9) | 38 | (23.9) |
| HECSI | | | | | | | | | | | | |
| n | 487 | | 325 | | 162 | | 472 | | 313 | | 159 | |
| Median (min-max) | 65.0 | (10-280) | 66.0 | (10-275) | 61.5 | (12-280) | 59.0 | (7-272) | 59.0 | (7-272) | 59.0 | (8-213) |
| DLQI | | | | | | | | | | | | |
| n | 479 | | 321 | | 158 | | 469 | | 310 | | 159 | |
| Median (min-max) | 12.0 | (0-30) | 12.0 | (0-30) | 12.0 | (2-30) | 11.0 | (1-30) | 11.0 | (1-28) | 11.0 | (2-30) |
| ≥4, n (%) | 453 | (94.6) | 305 | (95.0) | 148 | (93.7) | 452 | (96.4) | 299 | (96.5) | 153 | (96.2) |
| HESD itch (weekly | | | | | | | | | | | | |
| average) | | | | | | | | | | | | |
| n | 486 | | 324 | | 162 | | 469 | | 312 | | 157 | |
| Median (min-max) | 7.3 | (1.9-10.0) | 7.2 | (1.9-10.0) | 7.6 | (3.3-10.0) | 7.1 | (2.3-10.0) | 7.1 | (2.3-10.0) | 7.1 | (2.6-10.0) |
| ≥4, n (%) | 484 | (99.6) | 323 | (99.7) | 161 | (99.4) | 465 | (99.1) | 309 | (99.0) | 156 | (99.4) |
| HESD pain (weekly | | | | | | | | | | | | |
| average) | | | | | | | | | | | | |
| n | 486 | | 324 | | 162 | | 469 | | 312 | | 157 | |
| Median (min-max) | 7.1 | (0.8-10.0) | 7.1 | (0.8-10.0) | 7.2 | (0.9-10.0) | 6.7 | (0.0-10.0) | 6.7 | (0.0-10.0) | 6.7 | (1.0-10.0) |
| ≥4, n (%) | 440 | (90.5) | 291 | (89.8) | 149 | (92.0) | 435 | (92.8) | 294 | (94.2) | 141 | (89.8) |
| HESD (weekly | | | | | | | | | | | | |
| average) | | | | | | | | | | | | |
| n | 486 | | 324 | | 162 | | 469 | | 312 | | 157 | |
| Median (min-max) | 7.3 | (1.4-10.0) | 7.2 | (1.4-10.0) | 7.3 | (2.6-10.0) | 7.0 | (2.1-10.0) | 7.0 | (2.8-10.0) | 7.1 | (2.1-10.0) |
| ≥4, n (%) | 465 | (95.7) | 309 | (95.4) | 156 | (96.3) | 461 | (98.3) | 308 | (98.7) | 153 | (97.5) |
| CHE subtype,^{a} n (%) | | | | | | | | | | | | |
| Atopic hand eczema | 217 | (44.6) | 143 | (44.0) | 74 | (45.7) | 128 | (27.1) | 82 | (26.1) | 46 | (28.9) |
| Allergic contact | 84 | (17.2) | 51 | (15.7) | 33 | (20.4) | 49 | (10.4) | 27 | (8.6) | 22 | (13.8) |
| dermatitis | | | | | | | | | | | | |
| Hyperkeratotic | 77 | (15.8) | 57 | (17.5) | 20 | (12.3) | 129 | (27.3) | 86 | (27.4) | 43 | (27.0) |
| eczema | | | | | | | | | | | | |
| Irritant contact | 75 | (15.4) | 49 | (15.1) | 26 | (16.0) | 113 | (23.9) | 75 | (23.9) | 38 | (23.9) |
| dermatitis | | | | | | | | | | | | |
| Vesicular hand | 34 | (7.0) | 25 | (7.7) | 9 | (5.6) | 53 | (11.2) | 44 | (14.0) | 9 | (5.7) |
| eczema (pompholyx) | | | | | | | | | | | | |

Note:
Discrepancies in patient numbers for some assessments is due to some patients missing assessments at baseline; HESD itch, HESD pain, and HESD scores range from 0-10; HEIS and HEIS PDAL range from 0-4.
^{a}At baseline, one patient also presented with the CHE subtype contact urticaria/protein contact dermatitis.

TABLE 12
Summary of additional baseline characteristics and previous CHE treatments in DELTA 1 and DELTA 2

| DELTA 1 | | | DELTA 2 | | |
|---|---|---|---|---|---|
| | Delgocitinib | Cream | | Delgocitinib | Cream |
| Total | cream 20 mg/g | vehicle | Total | cream 20 mg/g | vehicle |
| (N = 487) | (N = 325) | (N = 162) | (N = 473) | (N = 314) | (N = 159) |

Number of flares experienced during the past year, n (%)

| 0 | 59 | (12.3) | 36 | (11.1) | 23 | (14.6) | 61 | (13.0) | 43 | (13.7) | 18 | (11.4) |
|---|---|---|---|---|---|---|---|---|---|---|---|---|
| 1-2 | 81 | (16.8) | 56 | (17.3) | 25 | (15.8) | 91 | (19.3) | 57 | (18.2) | 34 | (21.5) |
| 3-5 | 104 | (21.6) | 70 | (21.7) | 34 | (21.5) | 112 | (23.8) | 78 | (24.9) | 34 | (21.5) |
| 6-10 | 95 | (19.8) | 64 | (19.8) | 31 | (19.6) | 97 | (20.6) | 64 | (20.4) | 33 | (20.9) |
| 11-20 | 98 | (20.4) | 65 | (20.1) | 33 | (20.9) | 86 | (18.3) | 54 | (17.3) | 32 | (20.3) |
| >20 | 44 | (9.1) | 32 | (9.9) | 12 | (7.6) | 24 | (5.1) | 17 | (5.4) | 7 | (4.4) |
| HEIS | | | | | | | | | | | | |
| n | 479 | | 321 | | 158 | | 469 | | 310 | | 159 | |
| Median (min- | 2.6 | (0.0-4.0) | 2.6 | (0.0-4.0) | 2.7 | (0.0-4.0) | 2.4 | (0.2-4.0) | 2.4 | (0.4-4.0) | 2.4 | (0.2-4.0) |
| max) | | | | | | | | | | | | |
| HEIS PDAL | | | | | | | | | | | | |
| n | 479 | | 321 | | 158 | | 469 | | 310 | | 159 | |
| Median (min- | 2.7 | (0.0-4.0) | 2.7 | (0.0-4.0) | 2.7 | (0.0-4.0) | 2.7 | (0.0-4.0) | 2.7 | (0.0-4.0) | 2.7 | (0.0-4.0) |
| max) | | | | | | | | | | | | |

Previous CHE treatments

| | | | | | | | | | | | | |
|---|---|---|---|---|---|---|---|---|---|---|---|---|
| TCS, n (%) | | | | | | | | | | | | |
| Inadequate | 484 | (99.4) | 323 | (99.4) | 161 | (99.4) | 466 | (98.5) | 311 | (99.0) | 155 | (97.5) |
| response in the | | | | | | | | | | | | |
| last 12 months | | | | | | | | | | | | |
| Medically | 118 | (24.2) | 79 | (24.3) | 39 | (24.1) | 77 | (16.3) | 48 | (15.3) | 29 | (18.2) |
| inadvisable | | | | | | | | | | | | |
| TCI, n (%) | 174 | (35.7) | 121 | (37.2) | 53 | (32.7) | 175 | (37.0) | 113 | (36.0) | 62 | (39.0) |
| Phototherapy | 92 | (18.9) | 65 | (20.0) | 27 | (16.7) | 99 | (20.9) | 60 | (19.1) | 39 | (24.5) |
| and other | | | | | | | | | | | | |
| procedures, n | | | | | | | | | | | | |
| (%) | | | | | | | | | | | | |
| Oral retinoids, | 67 | (13.8) | 45 | (13.8) | 22 | (13.6) | 76 | (16.1) | 52 | (16.6) | 24 | (15.1) |
| n (%) | | | | | | | | | | | | |
| Oral | 59 | (12.1) | 46 | (14.2) | 13 | (8.0) | 78 | (16.5) | 50 | (15.9) | 28 | (17.6) |
| corticosteroids, | | | | | | | | | | | | |
| n (%) | | | | | | | | | | | | |
| Oral | 14 | (2.9) | 9 | (2.8) | 5 | (3.1) | 36 | (7.6) | 26 | (8.3) | 10 | (6.3) |
| methotrexate, | | | | | | | | | | | | |
| n (%) | | | | | | | | | | | | |
| Oral | 9 | (1.8) | 5 | (1.5) | 4 | (2.5) | 22 | (4.7) | 15 | (4.8) | 7 | (4.4) |
| cyclosporine, n | | | | | | | | | | | | |
| (%) | | | | | | | | | | | | |
| Other previous | 132 | (27.1) | 91 | (28.0) | 41 | (25.3) | 80 | (16.9) | 53 | (16.9) | 27 | (17.0) |
| CHE | | | | | | | | | | | | |
| treatments^{a}, n | | | | | | | | | | | | |
| (%) | | | | | | | | | | | | |

Note:
Discrepancies in patient numbers for some assessments is due to some patients missing assessments at baseline.
^{a}The most frequently reported (>2% of patients) included antihistamines, select emollients and protectives, and antibiotics.
CHE, Chronic Hand Eczema; HEIS, Hand Eczema Impact Scale; N, number of patients in analysis set; n, number of patients with observation; PDAL: Proximal Daily Activity Limitations; TCI, topical calcineurin inhibitors; TCS, topical corticosteroids.

### Investigational Medicinal Product (IMP)

The active and placebo IMP is set out in the table below:

| Investigational | Dosage | Active ingredient | Pack |
|---|---|---|---|
| medicinal product | form | and concentration | size |
| Delgocitinib cream | Cream | Delgocitinib, 20 mg/g | 15 g |
| 20 mg/g | | | |
| Cream vehicle | Cream | Vehicle | 15 g |

The delgocitinib cream 20 mg/g comprises:
Delgocitinib 20 mg/g; liquid paraffin, 100 mg/g; cetostearyl alcohol, 72 mg/g; macrogol cetostearyl ether, 18 mg/g; benzyl alcohol, 10 mg/g; citric acid
monohydrate, 1 mg/g; butylhydroxy anisole, 0.2 mg/g; disodium edetate, 0.6 mg/g; 3M hydrochloric acid, 17.7 mg/g; and purified water, 760 mg/g.

The cream vehicle comprises the same excipients in corresponding amounts.

### Administration of IMP

The IMP (delgocitinib cream 20 mg/g or cream vehicle) was applied as a topical application approximately 12 hours apart twice daily for 16 weeks.

### The Inclusion Criteria:

The subjects must fulfil all of the following criteria to be eligible for the trial:

1. Signed and dated informed consent has been obtained prior to any protocol-related procedures.

2. Age 18 years or above at screening.

3. Diagnosis of CHE, defined as hand eczema that has persisted for more than 3 months or returned twice or more within the last 12 months.

4. Disease severity graded as moderate to severe at screening and baseline according to IGA-CHE (i.e. an IGA-CHE score of 3 or 4).

5. HESD itch score (weekly average) of ≥4 points at baseline. The baseline weekly average will be calculated from daily assessments of itch severity during the 7 days immediately preceding the baseline visit (Day −7 to Day −1). A minimum of 4 itch scores out of the 7 days is required to calculate the baseline average score.

6. Subjects who have a documented recent history of inadequate response to treatment with TCS (at any time within 1 year before the screening visit) or for whom TCS are documented to be otherwise medically inadvisable (e.g. due to important side effects or safety risks).
Inadequate response is defined as a history of failure to achieve and maintain a low disease activity state (comparable to an IGA-CHE score of ≥2) despite treatment with a daily regimen of TCS of class III-IV (potent to very potent) for Europe and class IV-I (medium potency to very/ultra-high potency) for Canada, applied for at least 28 days or for the maximum duration recommended by the product prescribing information, whichever is shorter.
Important side effects or safety risks are those that outweigh the potential treatment benefits and include intolerance to treatment, hypersensitivity reactions, and significant skin atrophy as assessed by the physician.

7. Subjects adherent to standard non-medicated skin care including avoidance of known and relevant irritants and allergens.

8. A woman of childbearing potential must use an acceptable method of birth control throughout the trial up until the last application of IMP.

### Exclusion Criteria:

Subjects are not eligible for the trial if they fulfil any of the following criteria:

1. Concurrent skin diseases on the hands, e.g. tinea manuum.

2. Active AD requiring medical treatment in regions other than the hands and feet.

3. Active psoriasis on any part of the body.

4. Hyperkeratotic hand eczema in combination with a history of psoriasis on any part of the body.

5. Clinically significant infection (e.g. impetiginised hand eczema) on the hands.

6. Systemic treatment with immunosuppressive drugs (e.g. methotrexate, cyclosporine, azathioprine), immunomodulating drugs, retinoids (e.g. alitretinoin), or corticosteroids within 28 days prior to baseline (steroid eyedrops and inhaled or intranasal steroids corresponding to up to 1 mg prednisolone for allergic conjunctivitis, asthma, or rhinitis are allowed).

7. Use of tanning beds, phototherapy (e.g. UVB, UVA1, PUVA), or bleach baths on the hands within 28 days prior to baseline.

8. Previous or current treatment with JAK inhibitors (including delgocitinib/LEO 124249), systemic or topical.

9. Cutaneously applied treatment with immunomodulators (e.g. PDE-4 inhibitors, pimecrolimus, tacrolimus) or TCS on the hands within 14 days prior to baseline.

10. Use of systemic antibiotics or cutaneously applied antibiotics on the hands within 14 days prior to baseline.

11. Other transdermal or cutaneously applied therapy on the hands (except for the use of subject's own emollients) within 7 days prior to baseline.

12. Cutaneously applied treatments in regions other than the hands, which could interfere with clinical trial evaluations or pose a safety concern within 7 days prior to baseline.

13. Treatment with any marketed biological therapy or investigational biologic agents (including immunoglobulin, anti-IgE, and dupilumab):
Any cell-depleting agents including but not limited to rituximab: within 6 months prior to baseline, or until lymphocyte count returns to normal, whichever is longer.
Other biologics: within 3 months or 5 half-lives, whichever is longer, prior to baseline.

14. Treatment with any non-marketed drug substance (that is, an agent that has not yet been made available for clinical use following registration) within the last 28 days prior to baseline or 5 half-lives, whichever is the longest.

15. Clinically significant infection within 28 days prior to baseline which, in the opinion of the investigator, may compromise the safety of the subject in the trial, interfere with evaluation of the IMP, or reduce the subject's ability to participate in the trial.
Clinically significant infections are defined as:
   A systemic infection.
   A serious skin infection requiring parenteral (intravenous or intramuscular) antibiotics, antiviral, or antifungal medication.

16. History of any known primary immunodeficiency disorder including a positive HIV virus test at screening, or the subject taking antiretroviral medications as determined by medical history and/or subject's verbal report.

17. Major surgery within 8 weeks prior to screening, or planned in-patient surgery or hospitalisation during the trial period.

18. History of cancer:
Subjects who have had basal cell carcinoma, localised squamous cell carcinoma of the skin or in situ carcinoma of the cervix are eligible provided that the subject is in remission and curative therapy was completed at least 12 months prior to screening.
Subjects who have had other malignancies are eligible provided that the subject is in remission and curative therapy was completed at least 5 years prior to screening.

19. Any disorder which is not stable and could:
Affect the safety of the subject throughout the trial.
Impede the subject's ability to complete the trial.
Examples include but are not limited to cardiovascular, gastrointestinal, hepatic, renal, neurological, musculoskeletal, infectious, endocrine, metabolic, haematological, immunological, and psychiatric disorders, and major physical impairment.

20. Any abnormal finding which may:
Put the subject at risk because of their participation in the trial.
Influence the subject's ability to complete the trial.
The abnormal finding must be clinically significant and observed during the screening period. Examples include abnormal findings in physical examination, vital signs, ECG, haematology, clinical chemistry, or urinalysis.

21. Positive hepatitis B surface antigen or hepatitis C virus antibody serology at screening.

22. ALT or AST level ≥2.0×ULN at screening.

23. Known or suspected hypersensitivity to any component(s) of the IMP

24. Current participation in any other interventional clinical trial.

25. Previously randomised in this clinical trial.

26. Current or recent chronic alcohol or drug abuse, or any other condition associated with poor compliance as judged by the investigator.

27. Employees of the trial site, or any other individuals directly involved with the planning or conduct of the trial, or immediate family members of such individuals.

28. Subjects who are legally institutionalised.
Women who are pregnant or lactating.

### Trial Outline

Screening Period (Week −4 to Week 0) The screening period had a minimum duration of 1 week and a maximum duration of 4 weeks (i.e. screening visit took place between Week −4 and Week −1). For subjects using treatments that are listed as exclusion criteria, the duration of the screening period depends on the required wash-out period. In view of a 28-day wash-out for some of these treatments, the screening period can be extended up to 31 days. Only subjects who are considered able to stop prohibited treatment during the screening period without experiencing intolerable worsening of CHE symptoms will be included in the trial.

At the screening visit, the subjects' eligibility to enter the trial was checked.

The subjects' CHE subtype(s) was classified according to standard clinical practice in Canada and Europe.

### Treatment Period (Week 0 to Week 16)

At baseline (Day 1), the subjects' eligibility to enter the trial was confirmed. Eligible subjects was be randomised 2:1 to either delgocitinib cream 20 mg/g or cream vehicle.

The IMP (delgocitinib cream 20 mg/g or cream vehicle) was applied twice daily for 16 weeks. The first application of IMP occured at the trial site on Day 1 after all baseline assessments have been carried out. All subsequent IMP applications was done by the subjects at home.

During the 16-week treatment period, subjects returned to the trial sites for efficacy and safety assessments. The last IMP application occur at the subject's home before the subject attends the visit scheduled at Week 16. Efficacy and safety assessments during the treatment period was be performed.

All eligible subjects who do not permanently discontinue IMP prior to Week 16 was invited to participate in the LTE trial.

### Follow-Up Period (Week 16 to Week 18)

Those subjects not participating in the LTE trial a follow-up visit (performed via phone, but can be a site visit if needed) approximately 2 weeks after the last IMP application for assessment of safety. Note that for subjects who permanently discontinue IMP, the 2-week follow-up period will start at the time of last IMP application.

Subjects who will participate in the LTE trial will not be required to complete the follow-up period, as collection of safety data and safety surveillance will continue in the LTE trial.

### Efficacy at Week 4, 8 and 16:

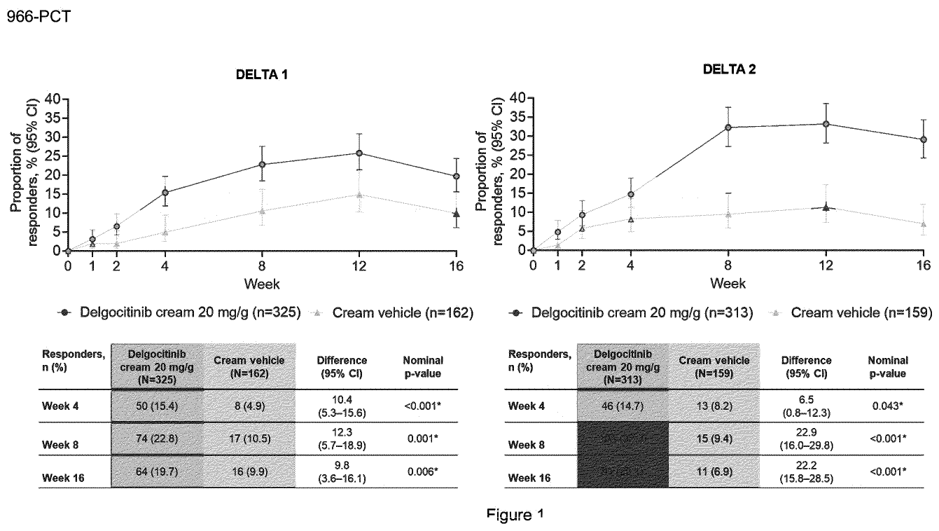

For DELTA 1 and DELTA 2, the primary study endpoint (IGA-CHE score of 0 [clear] or 1 [almost clear] with at least a 2-step improvement at Week 16) was achieved in 19.7% (n=64; DELTA 1) and 29.1% (n=91; DELTA 2) of patients in the delgocitinib cream groups, and 9.9% (n=16; DELTA 1) and 6.9% (n=11; DELTA 2) of those in the corresponding cream vehicle groups (FIG. 1). The proportion of patients achieving IGH-CHE TS at Week 16 was statistically significantly higher in delgocitinib cream groups compared with vehicle cream groups (P≤0.006 for both studies). Throughout the DELTA 1 and DELTA 2 16-week treatment periods, the proportion of patients achieving IGA-CHE treatment success was higher with delgocitinib cream treatment compared with cream vehicle, with statistically significantly more delgocitinib-treated patients achieving IGA-CHE treatment success versus those treated with cream vehicle at Weeks 4 (P≤0.043) and 8 (P≤0.001) (FIG. 1).

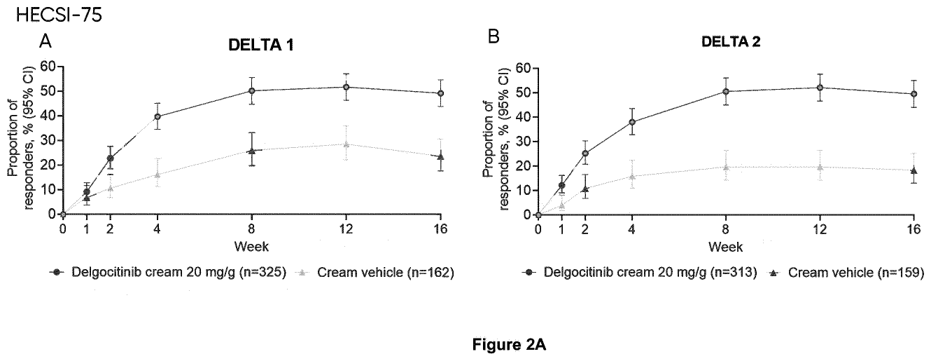

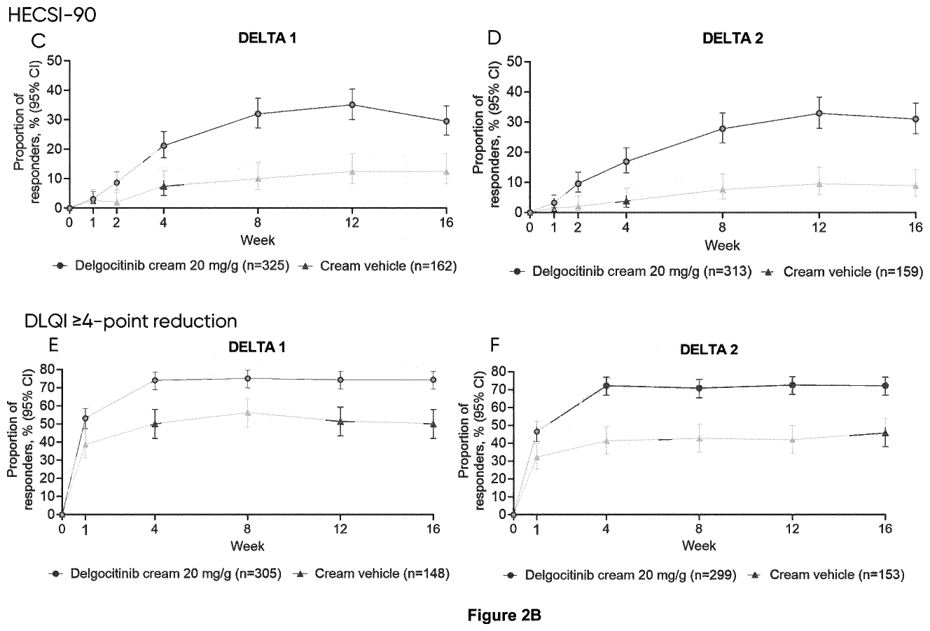

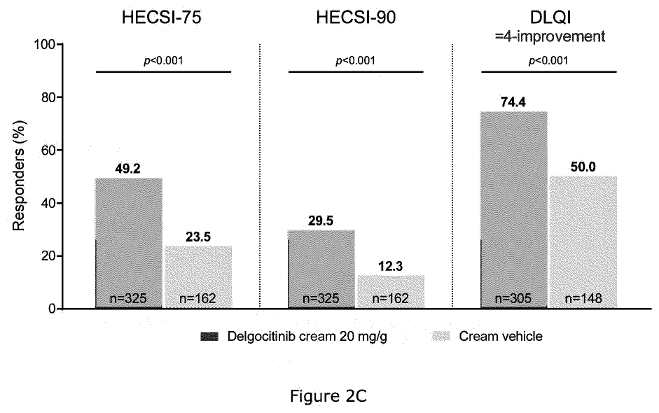

At Week 16, the percentage of patients achieving HECSI-75 and HECSI-90 scores, and ≥4-point reduction of DLQI score from baseline was statistically significantly higher in the DELTA 1 and DELTA 2 delgocitinib cream groups compared with their corresponding cream vehicle groups (P<0.001; FIG. 2A-C)

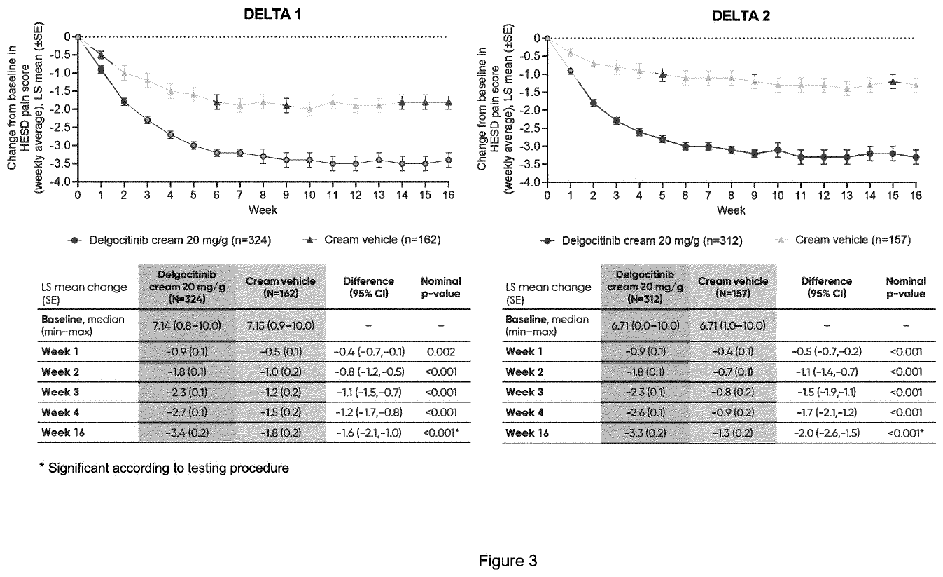

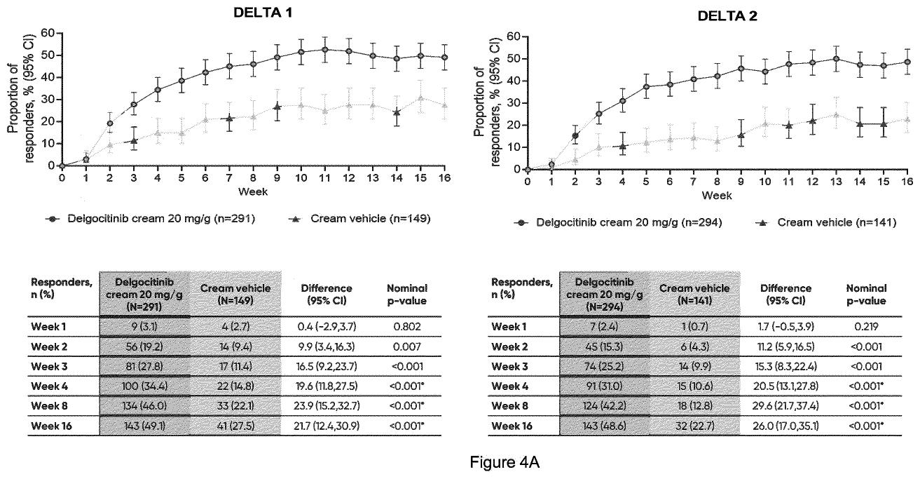

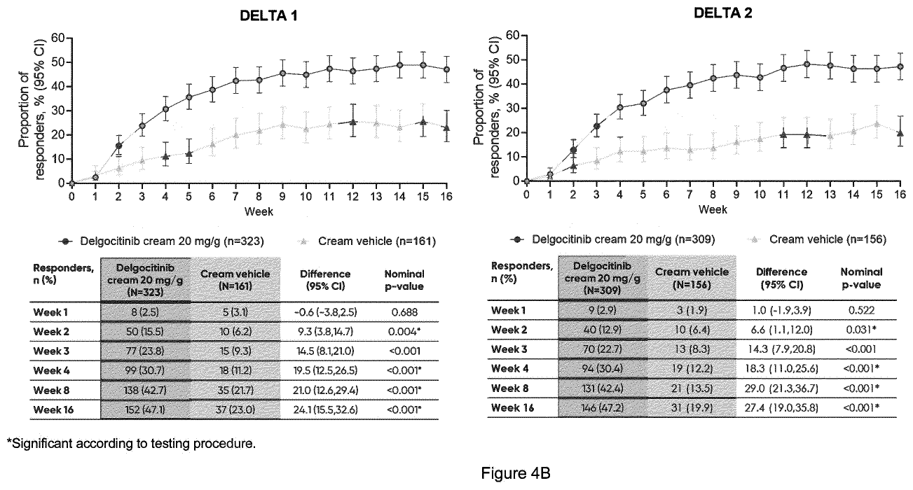

In the DELTA 1 trial, least squares (LS) mean change in HESD pain score from baseline to Week 16 was −3.4 in patients treated with delgocitinib cream and −1.8 in those treated with cream vehicle (P<0.001; FIG. 3). In the DELTA 2 study, corresponding LS mean changes were −3.3 in the delgocitinib cream group and −1.3 in the cream vehicle group (P<0.001). Among patients with a baseline HESD pain score of ≥4 points, the proportion of delgocitinib cream-treated patients achieving ≥4-point reduction of HESD pain score from baseline to Week 16 was statistically significantly higher (DELTA 1: 49.1%; DELTA 2: 48.6%) compared with those treated with cream vehicle (DELTA 1: 27.5%; DELTA 2: 22.7%; P<0.001; FIG. 4A-B).

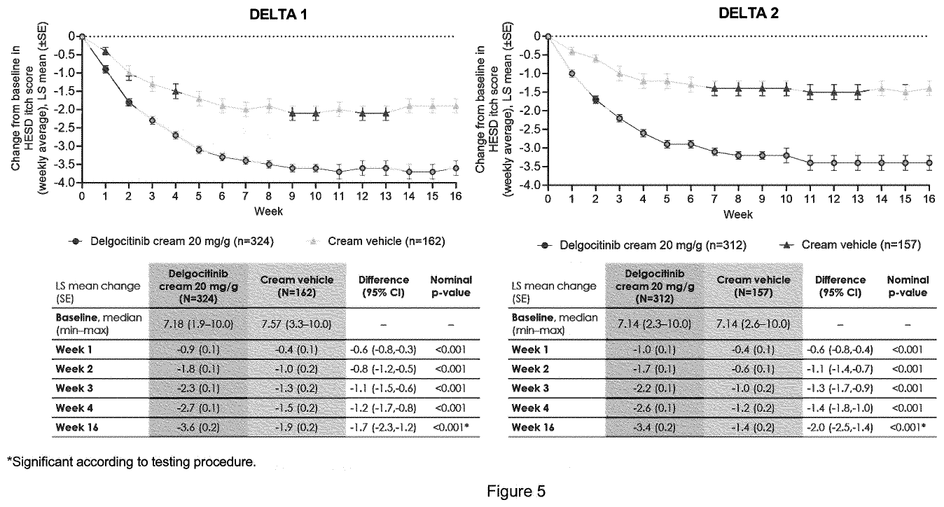

A similar reduction in HESD itch score was observed in DELTA 1, with LS mean change from baseline to Week 16 being −3.6 in the delgocitinib cream group and −1.9 in the cream vehicle group (P<0.001; FIG. 5). In the DELTA 2 study, corresponding LS mean changes were −3.4 for patients treated with delgocitinib cream and −1.4 for those treated with cream vehicle (P<0.001). Accordingly, among study participants with a baseline HESD itch score of ≥4 points, the proportion of delgocitinib cream-treated patients achieving ≥4-point reduction of HESD itch score from baseline to Week 16 was statistically significantly higher (DELTA 1: 47.1%; DELTA 2: 47.2%) compared with those treated with cream vehicle (DELTA 1: 23.0%; DELTA 2: 19.9%; delgocitinib cream vs cream vehicle: P<0.001; FIG. 4B).

In DELTA 1 and DELTA 2, delgocitinib-treated patients achieved significantly higher LS mean reductions in pain and itch scores from baseline versus those receiving cream vehicle at Weeks 1-4 (P≤0.002; FIGS. 3 and 5). Similarly, among patients with ≥4 points baseline HESD pain and itch scores, the proportion of patients treated with delgocitinib cream who achieved ≥4-point reduction of HESD pain and itch scores from baseline at Weeks 2-4 was significantly higher than those treated with cream vehicle (P≤0.031; FIG. 4A-B).

Among patients with a baseline HESD score of ≥4 points, the proportion of delgocitinib cream-treated patients achieving ≥4-point reduction of HESD score from baseline to Week 16 was statistically significantly higher (DELTA 1: 47.2%; DELTA 2: 44.5%) compared with those treated with cream vehicle (DELTA 1: 24.4%; DELTA 2: 20.9%; P<0.001. The difference from vehicle were 22.8% for Delta 1 and 23.6% for Delta 2.

### Safety:

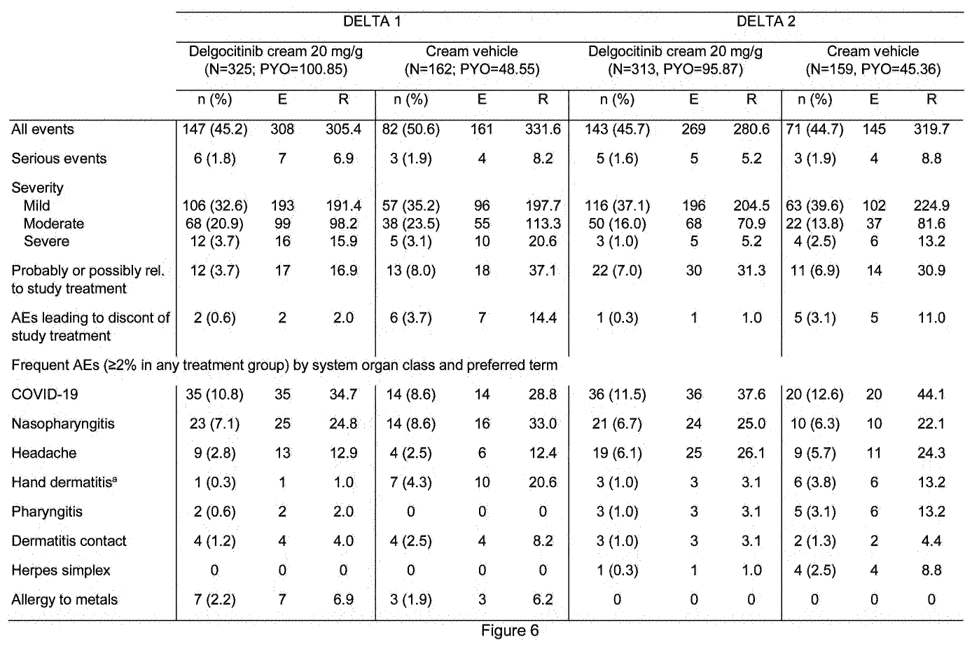

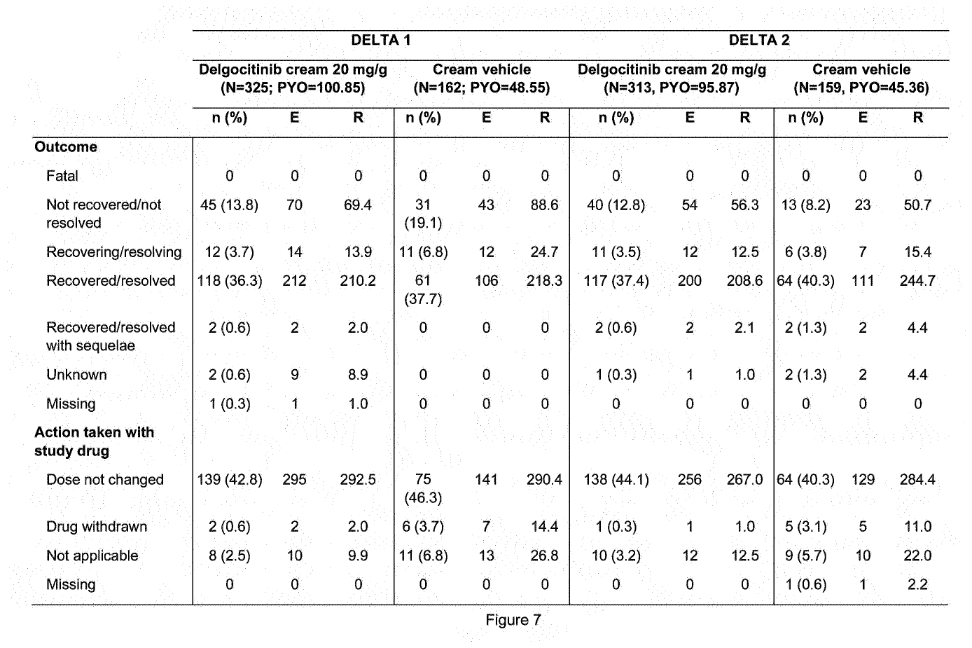

In DELTA 1 and DELTA 2, a comparable percentage of patients treated with delgocitinib cream and cream vehicle reported AEs (delgocitinib cream: 45.2% [DELTA 1] and 45.7% [DELTA 2]; cream vehicle: 50.6% [DELTA 1] and 44.7% [DELTA 2]; FIG. 6). Most AEs were mild to moderate and not considered related to study treatment. In both trials, most frequently reported AEs were COVID-19 and nasopharyngitis as well as headache. The proportion of patients reporting AEs leading to discontinuation of study treatment was lower in delgocitinib cream treatment groups (DELTA 1: 0.6%; DELTA 2: 0.3%) compared with their corresponding cream vehicle groups (DELTA 1: 3.7%; DELTA 2: 3.1%). Outcomes and action taken with study drug following occurrence of AEs were similar across all study groups in DELTA 1 and DELTA 2 (FIG. 7). In both studies, few serious AEs (≤1.9% of patients) were reported and all were assessed as not related to study drug by both study investigator and sponsor; none led to any safety concerns. No specific treatment-emergent safety concerns were identified in DELTA 1 and DELTA 2. No AEs of special interest (eczema herpeticum, deep vein thrombosis, or pulmonary embolism) were reported in DELTA 1 and DELTA 2. No major adverse cardiac events were reported in the delgocitinib treatment arms for both studies. There were no changes or differences between treatment groups in hematology, biochemistry, vital signs, physical examination, or ECG that were assessed to be of clinical relevance.

In DELTA 1 and DELTA 2, 41.9-47.2% of patients treated with delgocitinib cream and 36.9-41.2% of those treated with cream vehicle experienced no stinging or burning at Week 1, with the proportion of patients increasing to 90.0-91.7% (delgocitinib cream) and 88.6-91.8% (cream vehicle) at Week 16 (Table 13).

TABLE 13
Assessment of local tolerability^{a} for worst stinging or burning
in connection with study drug application weekly assessed by
study participants by selected weeks (safety analysis set)

| | DELTA 1 | | DELTA 2 | |
|---|---|---|---|---|
| | Delgocitinib | Cream | Delgocitinib | Cream |
| | 20 mg/g | vehicle | 20 mg/g | vehicle |
| n (%) | (N = 325) | (N = 162) | (N = 313) | (N = 159) |
| Week 1 | 288 (100.0) | 141 (100.0) | 248 (100.0) | 136 (100.0) |
| None | 136 (47.2) | 52 (36.9) | 104 (41.9) | 56 (41.2) |
| Mild | 94 (32.6) | 46 (32.6) | 93 (37.5) | 44 (32.4) |
| Moderate | 43 (14.9) | 23 (16.3) | 39 (15.7) | 22 (16.2) |
| Severe | 15 (5.2) | 20 (14.2) | 12 (4.8) | 14 (10.3) |
| Week 4 | 295 (100.0) | 143 (100.0) | 264 (100.0) | 137 (100.0) |
| None | 173 (58.6) | 61 (42.7) | 149 (56.4) | 64 (48.9) |
| Mild | 86 (29.2) | 50 (35.0) | 82 (31.1) | 38 (29.0) |
| Moderate | 30 (10.2) | 26 (18.2) | 25 (9.5) | 20 (15.3) |
| Severe | 6 (2.0) | 6 (4.2) | 8 (3.0) | 9 (6.9) |
| Week 8 | 293 (100.0) | 136 (100.0) | 252 (100.0) | 124 (100.0) |
| None | 207 (70.6) | 69 (50.7) | 154 (61.1) | 66 (53.2) |
| Mild | 58 (19.8) | 36 (26.5) | 71 (28.2) | 36 (29.0) |
| Moderate | 24 (8.2) | 28 (20.6) | 25 (9.9) | 14 (11.3) |
| Severe | 4 (1.4) | 3 (2.2) | 2 (0.8) | 8 (6.5) |
| Week 12 | 283 (100.0) | 134 (100.0) | 252 (100.0) | 119 (100.0) |
| None | 200 (70.7) | 70 (52.2) | 161 (63.9) | 73 (61.3) |
| Mild | 59 (20.8) | 41 (30.6) | 66 (26.2) | 28 (23.5) |
| Moderate | 21 (7.4) | 19 (14.2) | 22 (8.7) | 13 (10.9) |
| Severe | 3 (1.1) | 4 (3.0) | 3 (1.2) | 5 (4.2) |
| Week 16 | 303 (100.0) | 140 (100.0) | 291 (100.0) | 122 (100.0) |
| None | 278 (91.7) | 124 (88.6) | 262 (90.0) | 112 (91.8) |
| Mild | 18 (5.9) | 12 (8.6) | 22 (7.6) | 8 (6.6) |
| Moderate | 6 (2.0) | 4 (2.9) | 7 (2.4) | 2 (1.6) |
| Severe | 1 (0.3) | 0 | 0 | 0 |

^{a}Evaluated weekly in the eDiary.
N, number of patients in analysis set; n, number of patients with observation.

A numerically higher proportion of delgocitinib cream-treated patients reported no or mild tolerability issues compared to those in the cream vehicle groups from Week 1 and onwards. According to the investigator assessment of local tolerability, delgocitinib cream was well tolerated throughout both trials (Table 14). Few subjects had a patch test done during the study (DELTA 1: n=2 [delgocitinib cream] and n=4 [cream vehicle]; DELTA 2: n=1 [delgocitinib cream] and n=4 [cream vehicle]).

TABLE 14
Investigator assessment of local tolerability according
to suspected local skin reaction related to study
drug application by visit (safety analysis set)

| | DELTA 1 | | DELTA 2 | |
|---|---|---|---|---|
| | Delgocitinib | Cream | Delgocitinib | Cream |
| | 20 mg/g | vehicle | 20 mg/g | vehicle |
| n (%) | (N = 325) | (N = 162) | (N = 313) | (N = 159) |
| Week 1 | 318 (100.0) | 157 (100.0) | 303 (100.0) | 150(100.0) |
| Yes | 1 (0.3) | 3 (1.9) | 0 | 2 (1.3) |
| No | 317 (99.7) | 154 (98.1) | 303 (100.0) | 148 (98.7) |
| Week 2 | 311 (100.0) | 153 (100.0) | 294 (100.0) | 148 (100.0) |
| Yes | 0 | 0 | 0 | 1 (0.7) |
| No | 311 (100.0) | 153 (100) | 294 (100.0) | 147 (99.3) |
| Week 4 | 308 (100.0) | 152 (100.0) | 295 (100.0) | 136 (100.0) |
| Yes | 0 | 0 | 0 | 2 (1.5) |
| No | 308 (100.0) | 152 (100) | 295 (100.0) | 134 (98.5) |
| Week 8 | 308 (100.0) | 142 (100.0) | 289 (100.0) | 126 (100.0) |
| Yes | 0 | 1 (0.7) | 0 | 0 |
| No | 308 (100.0) | 141 (99.3) | 289 (100.0) | 126 (100.0) |
| Week 12 | 305 (100.0) | 143 (100.0) | 292 (100.0) | 123 (100.0) |
| Yes | 0 | 0 | 0 | 0 |
| No | 305 (100.0) | 143 (100) | 292 (100.0) | 123 (100.0) |
| Week 16 | 305 (100.0) | 141 (100.0) | 291 (100.0) | 122 (100.0) |
| Yes | 0 | 0 | 0 | 0 |
| No | 305 (100.0) | 141 (100) | 291 (100.0) | 122 (100.0) |
| Early | 18 (100.0) | 14 (100.0) | 17 (100.0) | 27 (100.0) |
| termination | | | | |
| Yes | 0 | 1 (7.1) | 1 (5.9) | 3 (11.1) |
| No | 18 (100.0) | 13 (92.9) | 16 (94.1) | 24 (88.9) |
| End of | 324 (100.0) | 157 (100.0) | 310 (100.0) | 150 (100.0) |
| treatment | | | | |
| Yes | 0 | 2 (1.3) | 1 (0.3) | 3 (2.0) |
| No | 324 (100) | 155 (98.7) | 309 (99.7) | 147 (98.0) |

Rates of all AEs, AEs related to study drug and AEs leading to discontinuation of study drug were numerically higher with cream vehicle compared to delgocitinib from baseline to trial completion

Overall, delgocitinib cream provided greater improvements in both patient- and clinician-reported efficacy outcomes versus cream vehicle and was well-tolerated over 16 weeks.

### Example 2

A phase 3 randomised, double-blind, vehicle-controlled, parallel-group, multi-site clinical trial to confirm efficacy and evaluate safety of twice-daily delgocitinib cream 20 mg/g compared with cream vehicle for a 16-week treatment period in adult subjects with moderate to severe chronic hand eczema (DELTA 2)

### Subjects

A total of 473 subjects were to be randomised 2:1 to delgocitinib cream 20 mg/g (314) or cream vehicle (159).

Inclusion and exclusion criteria are the same as in example 1.

Investigational medicinal product (IMP) and administration of IMP is the same as in example 1.

The trial design is the same as in example 1.

Results: At Week 16, a significantly greater proportion of delgocitinib-treated patients, compared to cream vehicle, achieved IGA-CHE TS (29.1% vs. 6.9%; p<0.001), HECSI-75 (49.5% vs. 18.2%; p<0.001), HECSI-90 (31.0% vs. 8.8%; p<0.001), and ≥4-point improvement in DLQI (72.2% vs. 45.8%; p<0.001). There was no difference between delgocitinib and cream vehicle in proportion of patients who reported adverse events (AEs; 45.7% vs. 44.7%) and serious AEs (1.6% vs. 1.9%). Rates of AEs assessed as probably or possibly related to study drug were consistent between delgocitinib (31.29 per 100-patient years of observation [PYO]) and cream vehicle (30.87 per 100 PYO). Rates of AEs leading to discontinuation of study drug were numerically higher with cream vehicle (11.02 per 100 PYO) compared to delgocitinib (1.04 per 100 PYO).

Overall, delgocitinib cream demonstrated greater improvements in both patient- and clinician-reported efficacy outcomes versus cream vehicle and was well-tolerated over 16 weeks. These results were consistent with those previously reported from the identically designed DELTA 1 study (example 1).

### Investigation of Systemic Exposure:

Blood samples collected 2-6 hours after application of the investigational medicinal product at Weeks 1, 4, and 16 were used to analyse plasma concentrations of delgocitinib using a liquid chromatography/mass spectrometry-based method with a lower limit of quantitation of 5 μg/ml. The inhibitory concentration of 50% (IC50) of delgocitinib was assessed using an in vitro IL-4 release assay in whole-blood of healthy volunteers (n=4). In the Phase 1 trial, single oral doses of delgocitinib (1.5, 3, 6, and 12 mg) were tested in healthy volunteers (n=40). Data are reported as geometric means.

Results: The DELTA 2 analysis included samples from 313 subjects on active treatment. The plasma concentration of delgocitinib was 0.21, 0.20 and 0.12 ng/ml at Weeks 1, 4 and 16, respectively. IC50 of delgocitinib was 17.2 ng/ml. In the Phase 1 study, the lowest tested oral dose of delgocitinib (1.5 mg) was perceived as a subtherapeutic dose. Peak systemic exposure (Cmax) of the 1.5 mg orally dosed delgocitinib was 7.2 ng/ml, meaning that systemic exposure after topical application in the DELTA 2 trial was ≥30-fold lower (7.2 ng/ml divided by 0.21 ng/ml).

Conclusion: Twice daily application of delgocitinib cream resulted in minimal systemic exposure, at least 80-fold below the whole-blood IC50 over 16 weeks (17.2 ng/ml divided by 0.21 ng/ml), and at least 30-fold below oral 1.5 mg delgocitinib dose with no overlap in plasma exposure between oral and topical administration. These data further support the favourable safety profile of topical delgocitinib cream and suggest that no systemic pharmacological effect is expected with 20 mg/g dosing in patients with moderate to severe CHE.

### Example 3

DELTA 1 and DELTA 2 were identically designed multicentre phase 3 trials in which adult patients with moderate-to-severe CHE were randomised (2:1) to double-blind treatment with delgocitinib cream 20 mg/g or cream vehicle twice daily for 16 weeks. In a pooled analysis of both trials, changes in EQ-5D scores from baseline to week 16 were assessed in all patients and in a post-hoc analysis of patients achieving clinical responses (defined as Hand Eczema Area and Severity Index (HECSI)-50, -75, and -90, Investigator's Global Assessment for CHE treatment success [IGA-CHE TS; clear or almost clear skin with a ≥2-step improvement from baseline] and ≥4-point improvements in Hand Eczema Symptom Diary (HESD) itch and pain scores.

### Results:

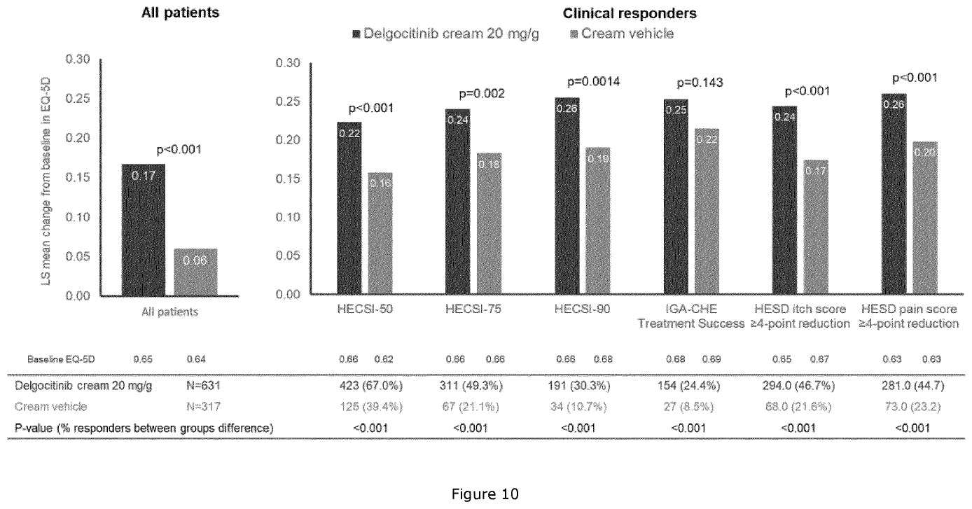

A total of 639 patients were randomised to delgocitinib cream 20 mg/g and 321 to cream vehicle across both trials. Most patients were white (90%), female (64%), with baseline moderate CHE disease severity (72%). Mean±SD EQ-5D score at baseline was 0.65±0.23 in the delgocitinib group and 0.64±0.24 in the cream vehicle group. Treatment with delgocitinib resulted in a significantly greater improvement in EQ-5D from baseline compared with cream vehicle at week 16 (least squares mean change [SE]0.17±0.01 vs. 0.06±0.01; difference [95% CI]0.11 (0.08, 0.13), p<0.001) (FIG. 10). Improvements in EQ-5D were significantly greater with delgocitinib versus cream vehicle across all five EQ-5D dimensions (mobility, self-care, usual activities, pain/discomfort and anxiety/depression). When analysed by clinical response, there were clinically meaningful changes in EQ-5D (minimally important difference >0.082) across the different responder thresholds, suggesting that improvements in HRQoL are likely associated with clinical response. Furthermore, improvements in EQ-5D were consistently higher with delgocitinib compared with cream vehicle within each clinical responder group, all of which were statistically significant except for the IGA-CHE TS category. This is in addition to significantly more patients treated with delgocitinib achieving a clinical response across different clinical responder thresholds.

### Conclusion

In patients with moderate-to-severe CHE with a substantial HRQoL impairment, 16 weeks of twice-daily treatment with delgocitinib cream 20 mg/g resulted in a statistically significantly greater improvements than cream vehicle in not just clinical outcomes, but also HRQoL as measured by the EQ-5D score. Among patients achieving a clinical response across various responder thresholds, treatment with delgocitinib resulted in an even greater HRQoL benefit than cream vehicle. This HRQoL improvement was considered clinically meaningful.

### Example 4

DELTA 1 and DELTA 2 were phase 3 trials of identical design. In both trials, adult patients with moderate-to-severe CHE were randomised (2:1) to double-blind treatment with delgocitinib cream 20 mg/g or cream vehicle twice daily for 16 weeks. Data were pooled from both trials and changes in DLQI scores were assessed from baseline to week 16 in all patients and in a post-hoc analysis of patients achieving clinical responses, defined as Hand Eczema Area and Severity Index (HECSI)-50, -75, and -90, Investigator's Global Assessment for CHE treatment success (IGA-CHE TS; a score of 0/1 with a ≥2-step improvement from baseline), and ≥4-point improvements in Hand Eczema Symptom Diary (HESD) itch and pain scores.

### Results:

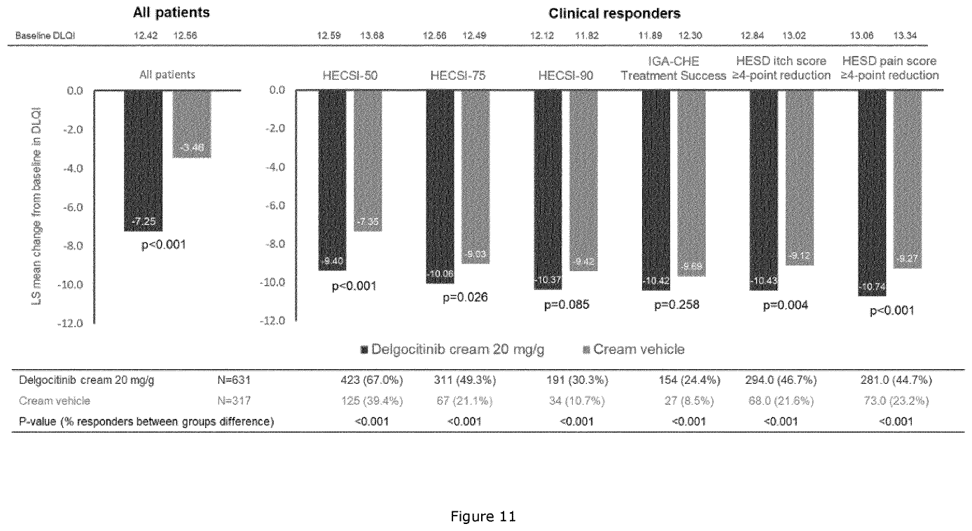

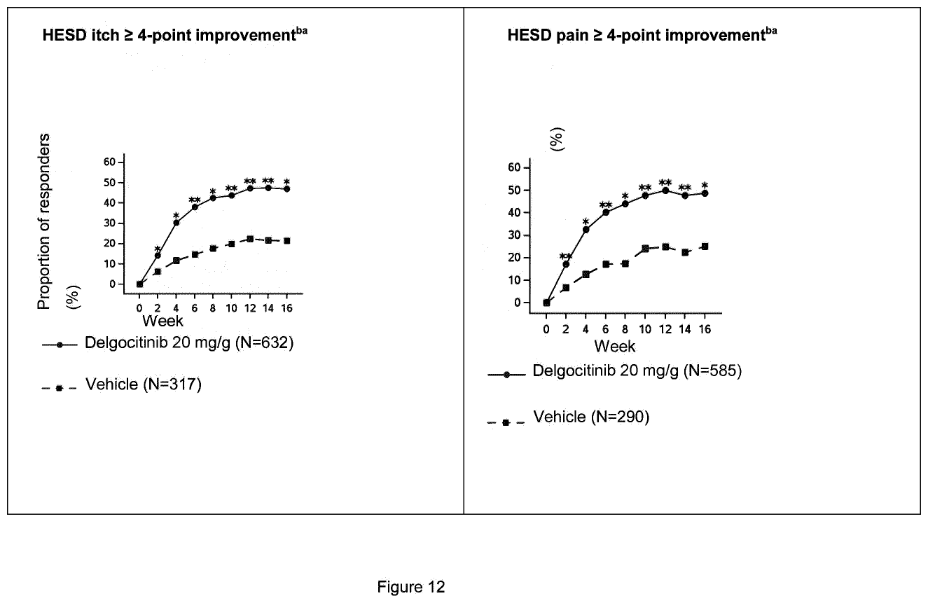

Across the two trials, 639 patients were randomised to delgocitinib cream 20 mg/g and 321 to cream vehicle. The majority of patients were white (90%), female (64%), and had a mean duration of CHE of approximately 10 years. Mean±SD DLQI score at baseline was 12.4±6.1 in the delgocitinib group and 12.4±6.7 in the cream vehicle group. Delgocitinib resulted in a significantly greater improvement (i.e., reduction) in DLQI from baseline compared with cream vehicle at week 16 (least squares mean change [SE]−7.25±0.22 vs. −3.46±0.31; difference [95% CI]−3.79 (−4.55, −3.04), p<0.001) (FIG. 11). Improvements in DLQI at week 16 were significantly greater (p<0.001) with delgocitinib versus cream vehicle across all ten DLQI items, in particular those concerning skin-related embarrassment or self-consciousness and effects on work, social and leisure, and other daily activities. When analysed by clinical response, greater improvements in DLQI with delgocitinib versus cream vehicle were observed across all clinical responder categories, with these differences statistically significant for all except HECSI-90 and IGA-CHE TS. This is in addition to a higher proportion of patients treated with delgocitinib than cream vehicle achieving a clinical response as assessed across all responder thresholds (all p<0.001).

### Conclusion

Twice-daily application of delgocitinib cream 20 mg/g resulted in a significant improvement in HRQoL versus cream vehicle as measured by DLQI in patients with moderate-to-severe CHE. In patients with a clinical response, the HRQoL benefit was greater with delgocitinib than with cream vehicle.

### Example 5

A phase 3 clinical trial to evaluate efficacy and safety of twice-daily applications of delgocitinib cream 20 mg/g compared with cream vehicle for a 16-week treatment period in adolescents 12-17 years of age with moderate to severe chronic hand eczema (DELTA TEEN).

### Subjects

About 92 subjects was randomised 3:1 to delgocitinib cream 20 mg/g or cream vehicle.

### Inclusion Criteria

The same as in example 2, except the following inclusion criteria:

1. Signed and dated IC has been obtained prior to any protocol-related procedures. Signed and dated IC must be provided by the subject's parent/guardian and/or by the subject in the form of a signed and dated IC/IA (as applicable according to national laws or regulation).

2. Age 12 to 17 years at screening and baseline.

3. HESD itch score (weekly average) of ≥4 points at baseline. The baseline weekly average will be calculated from daily assessments of itch severity during the 7 days immediately preceding the baseline visit (Day −7 to Day −1). A minimum of 4 itch scores out of the 7 days is required to calculate the baseline average score.

### Exclusion Criteria

The same as in example 1 except:

Subjects or parents/guardians who have a current or recent chronic alcohol or drug abuse, or any other condition associated with poor compliance as judged by the investigator.

### IMP

### Investigational Medicinal Product (IMP)

The active and placebo IMP is set out in the table below:

| | | | | Manufacturer |
|---|---|---|---|---|
| Investigational | Dosage | Active ingredient | Pack | responsible |
| medicinal product | form | and concentration | size | for batch release |
| Delgocitinib cream | Cream | Delgocitinib, | 30 g | LEO Pharma |
| 20 mg/g | | 20 mg/g | | A/S |
| Cream vehicle | Cream | Vehicle | 30 g | LEO Pharma |
| | | | | A/S |

Excipients of delgocitinib cream 20 mg/g and cream vehicle is the same as in example 1.

The IMP (delgocitinib cream 20 mg/g or cream vehicle) was applied as a topical application approximately 12 hours apart twice daily for 16 weeks.

### Trial Outline

The trial is a phase 3, randomised, double-blind, vehicle-controlled, parallel-group, multi-site trial. The trial is designed to evaluate the efficacy and safety of delgocitinib cream 20 mg/g applied twice-daily for 16 weeks in adolescents 12-17 years of age with moderate to severe chronic hand eczema.

### Screening Period (Week −4 to Week 0)

The screening period has a minimum duration of 1 week and a maximum duration of 4 weeks (i.e. screening visit should take place between Week −4 and Week −1). For subjects using treatments that are listed as exclusion criteria, the duration of the screening period will depend on the required wash-out period. In view of a 28-day wash-out for some of these treatments and the allowed visit window (+3 days), the screening period was extended up to 31 days. Only subjects who are considered able to stop prohibited treatment during the screening period without experiencing intolerable worsening of CHE signs and symptoms will be included in the trial.

At the screening visit, the subjects' eligibility to enter the trial was checked. Trial-specific measurements will be performed as outlined in the schedule of trial procedures).

The subjects' CHE subtype(s) will be classified according to standard clinical practice in Europe, Canada, and Australia.

### Treatment Period (Week 0 to 16)

At baseline (Day 1), the subjects' eligibility to enter the was confirmed. Eligible subjects was randomised 3:1 to either delgocitinib cream 20 mg/g or cream vehicle.

Subjects/caregivers applied the IMP (delgocitinib cream 20 mg/g or cream vehicle) twice-daily for 16 weeks. The first application of IMP occurred at the trial site at baseline (Day 1) after all baseline assessments have had carried out. All subsequent IMP applications was performed by the subjects/caregivers at home.

During the 16-week treatment period, subjects returned to the trial sites for efficacy and safety assessments.

### Follow-Up Period (Week 16-18)

Subjects was followed-up via phone (can be a site visit) approximately 2 weeks after the last IMP application for assessment of safety. Note that for subjects who discontinued treatment with IMP prematurely, the 2-week follow-up period started at the time of last IMP application.

### Endpoints

Endpoints for the trial are disclosed in table 9 and 10 above.

### Example 6

Two different formulations comprising Delgocitinib were tested in
I) Human skin explant (NativeSkin) biomarker testing and
II) Open Flow Microperfusion (OFM) in vivo in pigs, to explore the PKPD profiles of delgocitinib in the different test systems.

### Formulations Tested

Ointment: 30 mg/g Delgocitinib ointment as disclosed in D1 (WO2017/125523)
Cream: 20 mg/g Delgocitinib cream formulation as disclosed in the patent application as filed (WO2020229622)

### Human Skin Explant Biomarker Testing

The two formulations were tested in NativeSkin models from Genoskin, France (LEO Pharma A/S experiment number EXP-2017-6909). The biomarkers for JAK-inhibition, phosphorylated STAT3/total STAT3 (pY-STAT3/STAT3) ratio was measured at 24 hours after application of either ointment (30 mg delgocitinib/g) or cream (20 mg delgocitinib/g), compared to control cream and blank (3 different donors for each treatment).

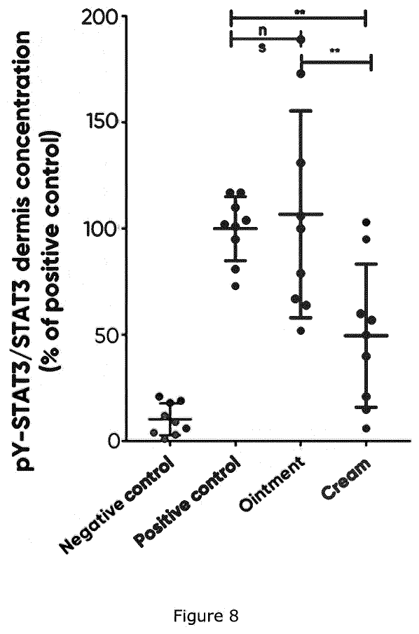

The results are shown in FIG. 8.

The cream formulation of Delgocitinib inhibited the upregulation of pY-STAT3/STAT3 ratio significantly more compared to the ointment (FIG. 3) showing that significantly more Delgocitinib permeates into the skin from the 20 mg/g cream of the invention compared to the 30 mg/g ointment described in WO2017/125523.

### Open Flow Micro-Perfusion

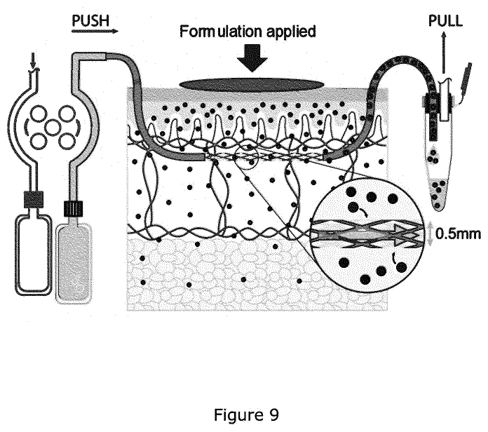

The two formulations were tested in vivo in landrace pigs at Joanneum Research Institute, Austria (LEO Pharma A/S report number REP-DJR-2018-01). Delgocitinib concentrations were measured in dermis up to 12 hours after application of either ointment (30 mg delgocitinib/g) or cream (20 mg delgocitinib/g), by using OFM (FIG. 9).

The unbound concentration of delgocitinib in dermal interstitial fluid was calculated from perfusate concentration compensated for relative recovery in the set-up and protein binding measured in vitro. Two pigs with two test fields for each formulation were treated and three probes were inserted at each test field (n=12 individual probes per treatment).

The unbound dermal interstitial fluid (dISF) concentration of delgocitinib was higher after application of the cream formulation compared to the ointment (FIG. 3). The dISF concentration after application of the cream was clearly higher compared to the unbound potency, showing that more Delgocitinib permeates into the skin from the 20 mg/g cream compared to the 30 mg/g ointment, which was in line with the inhibitory effect on biomarker seen in human skin explants experiment.

### Example 7

Itch and pain are two of the most common and burdensome symptoms of chronic hand eczema. Delgocitinib cream, a topical pan-Janus kinase inhibitor, was well tolerated and demonstrated significant improvement in all primary and secondary efficacy endpoints in DELTA-1 and DELTA-2.

This analysis includes pooled data from DELTA-1 and -2 (delgocitinib cream 20 mg/g [n=639]; cream vehicle [n=321]; twice-daily). The Hand Eczema Symptom eDiary (HESD) captured patient-reported severity of itch and pain over the past 24 hours on an 11-point numeric rating scale (0=no itch/pain to 10=severe itch/pain). Changes in itch and pain from baseline were assessed daily during Week (W)1 and weekly from W1-16.

For itch, least square (LS) mean reduction from baseline was 0.22-1.32 in the delgocitinib cream group and 0.21-0.68 in the cream vehicle group between Day (D)1-7. For pain, an LS mean reduction from baseline of 0.13-1.43 versus 0.26-0.81 was observed, respectively. From W1-16, delgocitinib-treated patients achieved higher LS mean reductions from baseline in itch score (1.0-3.5) versus those receiving cream vehicle (0.4-1.7; P<0.001). Similar results were seen for LS mean pain reduction (delgocitinib cream: 0.9-3.3; cream vehicle: 0.4-1.5; P<0.001). Among patients with ≥4 points baseline HESD itch/pain score, the proportion of delgocitinib cream-treated patients achieving ≥4-point reduction of HESD itch/pain score from baseline at W2-16 was higher than those treated with cream vehicle (P≤0.001).

Early onset of itch and pain reduction was observed within W1 for delgocitinib-treated patients, with reductions remaining significantly greater versus cream vehicle-treated patients from W1 to W16.

### Example 8

In the DELTA 2 phase 3 trial, delgocitinib cream 20 mg/g, a topical pan-Janus kinase inhibitor, was well-tolerated and demonstrated significant improvement in all efficacy endpoints versus cream vehicle in adults with moderate to severe chronic hand eczema (CHE).

Pharmacokinetic blood sampling in DELTA 2 was performed 2-6 hours after delgocitinib application at Weeks 1, 4, and 16 using a liquid chromatography/mass spectrometry-based method (lower limit of quantitation: 5 μg/ml). In the phase 1 trial (NCT05050279), single oral doses of delgocitinib were tested in healthy volunteers with sampling performed for up to 24-hours post-administration.

In Delta 2, the geometric mean+/−SD maximum plasma concentration (Cₘₐₓ) and area under the concentration-curve from time 0 to 12 hours (AUC₀₋₁₂) on day 8 was 0.46 ng/ml+/−0.28 and 3.7ng*h/ml+/−1.88, respectively. Steady state was reached by Day 8. The systemic exposure (AUC and Cₘₐₓ) between day 1 and day 8 were similar and negligible.

In DELTA 2, minimal systemic exposure was recorded in 313 delgocitinib-treated patients, with the highest geometric mean plasma concentration being 0.21 ng/ml at Week 1 (n=286). By week 16 systemic absorption was detected only in some patients and the detectable absorption had decreased by 48% to a mean of 0.11 ng/ml in the remaining subjects. In the Phase 1 trial, the lowest oral delgocitinib dose tested (1.5 mg; n=8) is regarded as subtherapeutic and showed a peak systemic exposure (geometric mean Cmax) of 7.2 ng/ml. In DELTA 2, adverse events (AEs) were reported by 45.7% (n=143/313; delgocitinib cream) and 44.7% (n=71/159; cream vehicle) of patients, with COVID-19 being most common (11.5% vs 12.6%, respectively). The rate of possibly or probably related AEs was low and similar between delgocitinib cream and cream vehicle. No deaths were reported. Few SAEs were reported with none assessed as related to the study drug.

Plasma protein binding of delgocitinib is 22 to 29%.

Following repeated topical administration of delgocitinib cream, the average half-life of delgocitinib was estimated to be 20.3 hours.

The DELTA 2 trial demonstrated minimal systemic exposure in association with a favorable safety profile, supporting a lack of meaningful systemic effect from twice-daily applications of delgocitinib cream in patients with moderate to severe CHE.

1. A method of treating moderate to severe chronic hand eczema in a human patient in need thereof comprising twice daily administration to the skin of said human patient of an acidified aqueous topical composition comprising comprises 20 mg/g delgocitinib on a free base basis or a pharmaceutically acceptable salt thereof, wherein the patient has disease severity graded as moderate to severe at screening and baseline according to IGA-CHE (i.e. an IGA-CHE score of 3 or 4).

2. The method of claim 1, wherein the patient has a HESD itch score (weekly average) of 4 points at baseline.

3. The method of claim 1, wherein the patient is an adolescent aged 12 to 17 or an adult aged 18 years or above; wherein the patient have hand eczema that has persisted for more than 3 months or returned twice or more within the last 12 months.

4. The method of claim 1, wherein the patient has a documented recent history of inadequate response to treatment with topical corticosteroids (TCS) or for whom TCS are documented to be otherwise medically inadvisable (due to important side effects or safety risks).

5. The method of claim 1, wherein the patient achieves an IGA-CHE score of 0 (clear) or 1 (almost clear) with a >2-step improvement from baseline at week 1, week 2, week 4, week 8, and/or week 16.

6-9. (canceled)

10. The method of claim 1, wherein the patient achieves at least 90% improvement in HECSI score from baseline at week 16, or week, 8 or week 4, week 2 or week 1.

11. The method of claim 1, wherein the patient achieves at least 75% improvement in HECSI score from baseline at week 16, or week 8, or week 4, or week 2 or week 1.

12. The method of claim 1, wherein the patient has a baseline HESD score (weekly average) >4 points and achieves a reduction of HESD score (weekly average) of >4 points from baseline at week 1, week 2, week 4, week 8, week 12, and/or week 16.

13-17. (canceled)

18. The method of claim 1, wherein the patient has a baseline HESD itch score (weekly average) >4 points and achieves a reduction of HEDS itch score (weekly average) of >points from baseline at week 1, week 2, week 4, week 8 and/or week 16.

19-27. (canceled)

28. The method of claim 1, wherein the patient achieves a reduction of DLQI score of >4 points from baseline at week 1, week 2, week 4, and/or week 16.

29-32. (canceled)

33. The method of claim 1, wherein no accumulation of delgocitinib in the body is observed.

34. The method of claim 1, wherein the administration to the skin of a human patient is an administration to the hands and wrists of a human patient.

35. The method of claim 1, wherein the treatment is continued until the patient achieves clear or almost clear skin, and wherein in the event of recurrence of the signs and symptoms of flares, twice daily treatment of the affected areas is re-initiated as needed.

36. (canceled)

37. The method of claim 1, wherein delgocitinib is dissolved in the aqueous phase of the topical formulation.

38. The method of claim 1, wherein the aqueous topical formulation has a pH below about 4.6 or below about 4.4.

39-41. (canceled)

42. The method of claim 1, wherein the aqueous topical formulation comprises a lipid base.

43. (canceled)

44. The method of claim 42, wherein the lipid base is liquid paraffin.

45. The method of claim 1, wherein the aqueous topical formulation has a pH between about 3.8 and about 4.6.

46. The method of claim 1, wherein the aqueous topical formulation has a pH of about 4.3 or below.

47. The method of claim 1, wherein the aqueous topical formulation has a pH of about 4.2 or below.
